(19) United States
(12) Patent Application Publication
(10) Pub. No.: US 2015/0343016 A1
(43) Pub. Date: Dec. 3, 2015

(54) CHEMICAL ENTITIES AND THERAPEUTIC USES THEREOF

(71) Applicant: The Regents of the University of Colorado, a body corporate, Denver, CO (US)

(72) Inventors: Tin Tin Su, Boulder, CO (US); Gan Zhang, Niwot, CO (US)

(21) Appl. No.: 14/582,981

(22) Filed: Dec. 24, 2014

Related U.S. Application Data
(63) Continuation of application No. 13/988,501, filed on Nov. 12, 2013, now abandoned.
(60) Provisional application No. 61/419,736, filed on Dec. 3, 2010.

(51) Int. Cl. A61K 38/12; A61N 5/10; A61K 31/704; A61K 45/06; A61K 31/337

(52) U.S. Cl. CPC A61K 38/12; A61K 45/06; A61K 31/337; A61K 31/704; A61N 5/10

(57) ABSTRACT

The present invention describes the use of translation inhibitors for the treatment of cancer and other disorders. Described herein are translation-inhibiting compounds, and methods of using those compounds for the treatment of cancer and other disorders. In some aspects the compounds inhibit translation elongation at the ribosome. In some aspects the compounds are used alone or in combination with known therapies.

### CROSS-REFERENCE TO RELATED APPLICATIONS

This application claims the benefit of U.S. Provisional Application No. 61/419,736, entitled “Chemical Entities And Therapeutic Uses Thereof,” filed on Dec. 3, 2010, which is incorporated herein by reference in its entirety.

### STATEMENT AS TO FEDERALLY SPONSORED RESEARCH

This invention was made with government support under GM664441, DE017494, GM 073973, and GM087276 awarded by the National Institutes of Health (NIH). The US government has certain rights in the invention.

### BACKGROUND OF THE INVENTION

Current cancer therapies target every aspect of cancer cell growth and division except translation of RNA into protein by the ribosome. Increased ribosome activity and protein translation however, is a hallmark of cancer cells and is required for disease progression. FIG. 1 depicts current targets for cancer therapies (arrowheads).

The ribosome is not typically a target for cancer therapy. While ribosome activity and protein translation can be regulated by blocking the elongations step of translation at the ribososome using “anti-ribosomals,” these anti-ribosomals have been omitted from cancer therapies as a result of two strong, long-standing biases. First, translation inhibitors are thought to lack the specificity required to target cancer cells. Second, translation inhibitors are thought to be too toxic to be used as therapeutics. For example, Tobey et al. concluded that bouvardin, a translation inhibitor, “ . . . does not appear to possess the type of properties normally associated with a useful chemotherapeutic agent” (Tobey et al., CANCER RESEARCH 38, 4415-4421, December 1978).

### SUMMARY OF THE INVENTION

The present invention provides the use of translation inhibitors for the treatment of cancer and other disorders. Described herein are translation-inhibiting compounds, and methods of using those compounds for the treatment of cancer and other disorders. In some aspects the compounds inhibit translation elongation at the ribosome. In some aspects the compounds are used alone or in combination with known therapies.

In one aspect, the invention provides a compound of Formula I and its pharmaceutically acceptable salts thereof. FIG. 2 illustrates the formula (I) compound of the invention.

In one aspect, the invention provides a compound of Formula II and its pharmaceutically acceptable salts thereof. FIG. 3 illustrates the formula (II) of compound of the invention.

In one aspect, the invention provides compounds of Formula III, depicting family of bouvardin derivatives, which differ from bouvardin in that they lack bouvardin's hexapeptide ring, and pharmaceutically acceptable salts thereof. FIG. 4 illustrates the general Formula (III) of compounds of the invention. In some embodiments, a compound of Formula III is the compound wherein R1=CH₂OH, R2=OMe, R3=H R4=H and R5=H.

In some embodiments, compounds of Formula I, Formula II or Formula III inhibit translation elongation at the ribosome.

In one aspect, the invention provides a pharmaceutical composition comprising a pharmaceutically acceptable carrier and a compound of Formula I, Formula II, or Formula III. In some embodiments, the pharmaceutical composition is formulated as a solid, semi-solid, liquid, or aerosol dosage form.

In one aspect, the invention provides a method of modulating the activity of a ribosome comprising contacting said ribosome with an effective amount of a compound of Formula I, Formula II or Formula III.

In one aspect, the invention provides a method of treating a condition or disorder mediated by abnormal protein synthesis in a subject in need of such treatment comprising administering to a mammal in need thereof a therapeutically effective amount of a compound of Formula I, Formula II or Formula III. In some embodiments, the ribosome related disorder is a cancer, bone disorder, inflammatory disease, immune disease, nervous system disease, respiratory disease, or cardiac disease.

In some embodiments of the methods of the invention, the method further comprises the step of administering a second therapeutic agent.

In some embodiments the inhibitor of translation is an inhibitor of translation elongation at the eukaryotic ribosome.

In some embodiments the invention provides method for treating a disorder in a subject comprising:administering to the subject an effective amount of an inhibitor of protein translation, and administering to the subject an effective amount of a chemotherapeutic composition.

In some embodiments the invention provides a method for treating a disorder in a subject comprising: administering to the subject an effective amount of an inhibitor of protein translation, and administering to the subject an effective amount of radiation therapy.

In some embodiments the invention provides a method for treating a disorder in a subject comprising administering to the subject a modulator of protein translation.

In some embodiments the modulator or inhibitor of protein translation is bouvardin, streptovitacin A, or a derivative of bouvardin.

In some embodiments the derivative of bouvardin is: the compound of formula III wherein R1 is a CH₂OH or COO-lower alkyl (C1-C6) group, R2 is O-lower alkyl (C1-C6), R3 is H or lower alkyl (C1-C6), R4 is H or a protecting group, and R5 is H or a lower alkyl (C1-C6).

In some embodiments the protecting group is BOC or CBZ.

In some embodiments the structures below are not the derivative of bouvardin:

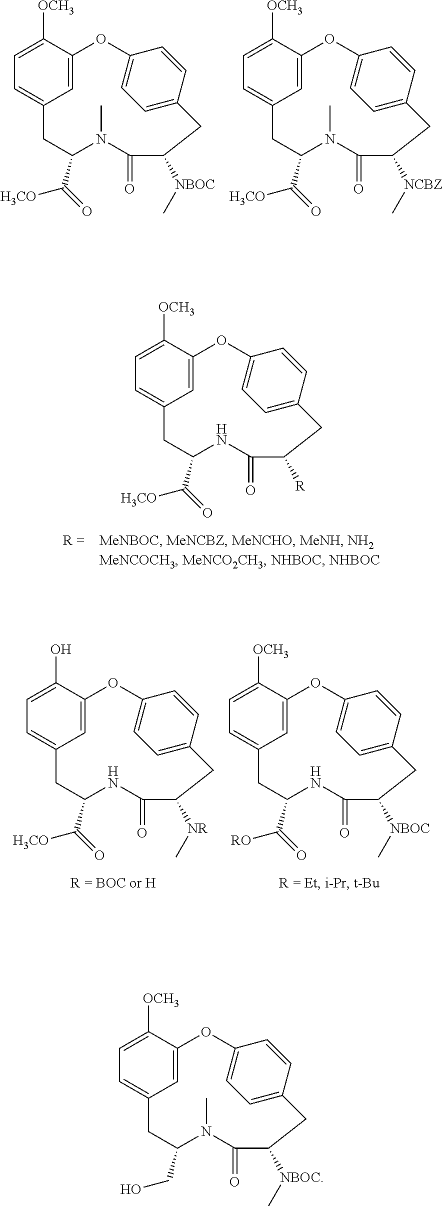

In some embodiments the structures below are not the derivative of bouvardin:

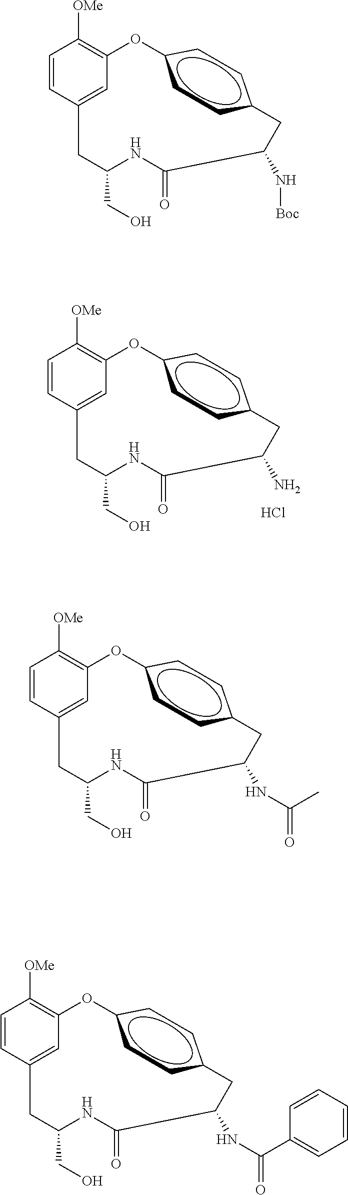

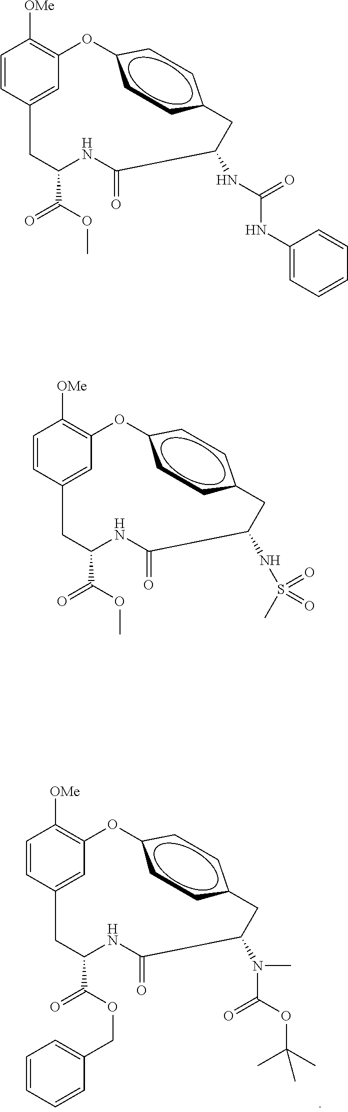

In some embodiments the structures below are not the derivative of bouvardin:

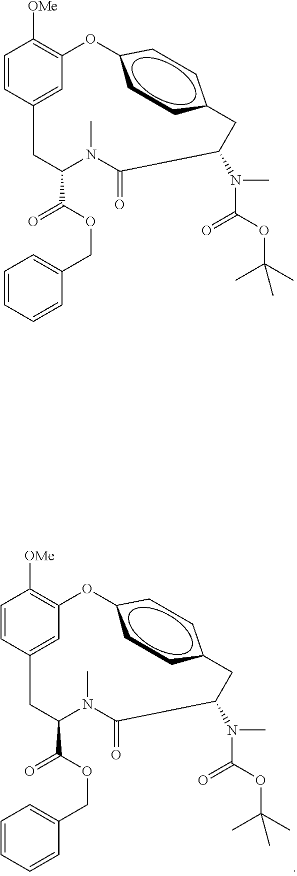

In some embodiments R1=COOCH₃; R2=OCH₃; R3=H; R4=Boc; R5=CH₃. ISO R1=CH₂OH, R2=OMe, R3=H, R4=H and R5=H. In some embodiments R1=CH₂OH, R2=OMe, R3=H, R4=BOC and R5=H.

ISO derivative of bouvardin is:

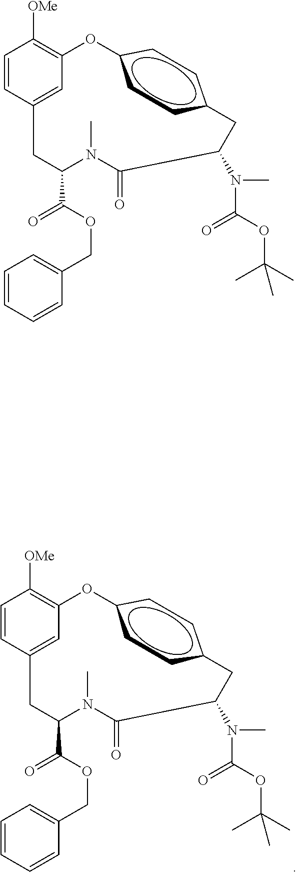

In some embodiments the disorder is cancer. ISO cancer is in tissues of head and neck. ISO cancer is in tissues of the lung. In some embodiments the cancer is of the lymphatic system. In some embodiments disorder is an immune disorder. In some embodiments the disorder is diabetes. In some embodiments the disorder is a neurological disorder associated with abnormal protein accumulation.

In some embodiments the radiation therapy comprises exposing the subject to ionizing radiation. In some embodiments the radiation therapy comprises exposing the subject to particles from a radioactive substance. In some embodiments the radiation therapy comprises exposing the subject to radiation from an external source. In some embodiments the radiation therapy is for curative, adjuvant, neoadjuvant, therapeutic or palliative purposes. In some embodiments the radiation therapy is given at a dosage of 20 Gy to 80 Gy total, fractionated into smaller doses over a course of treatment that may last several weeks.

In some embodiments the subject is a mammal. In some embodiments subject has been diagnosed with cancer.

In some embodiments the chemotherapy composition comprises a taxane. In some embodiments the taxane is paclitaxel. In some embodiments the effective dose of a taxane is less when used in combination with the compositions of the invention than the effective dose of taxane alone. In some embodiments the taxane and the inhibitor of protein translation are combined in a single formulation.

In some embodiments chemotherapy composition comprises a platinum-based chemotherapy drug. In some embodiments chemotherapy composition comprises doxorubicin or a derivative of doxorubicin.

In some embodiments the inhibitor of protein translation is administered orally, intravenously, or by local injection. In some embodiments a composition of the invention is administered orally, intravenously, or by local injection.

In some embodiments the inhibitor of protein translation is administered at a concentration of 0.01 to 10 mg/kg. In some embodiments a compound of the invention is administered at a concentration of 0.01 to 10 mg/kg.

In some embodiments the invention provides a method comprising composing instruction for the use of an inhibitor of translation to be used in combination with a chemotherapeutic agent or radiation therapy wherein the instructions are given to a subject with an inhibitor of translation compound.

In some embodiments the invention provides pharmaceutical composition comprising a compound:

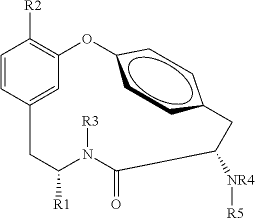

wherein R1 is a CH₂OH or C(O)O-lower alkyl (C1-C6) group, R2 is O-lower alkyl (C1-C6) group, R3 is H or lower alkyl (C1-C6), R4 is H or a protecting group, and R5 is H or a lower alkyl (C1-C6) group. In some embodiments the invention provides the protecting group is BOC or CBZ. In some embodiments the invention provides R1=COOCH₃; R2=OCH₃; R3=H; R4=Boc; R5=CH₃. In some embodiments the invention provides R1=CH₂OH, R2=OMe, R3=H, R4=H and R5=H. In some embodiments the invention provides R1=CH₂OH, R2=OMe, R3=H, R4=BOC and R5=H.

In some embodiments the pharmaceutical composition excludes the structures below:

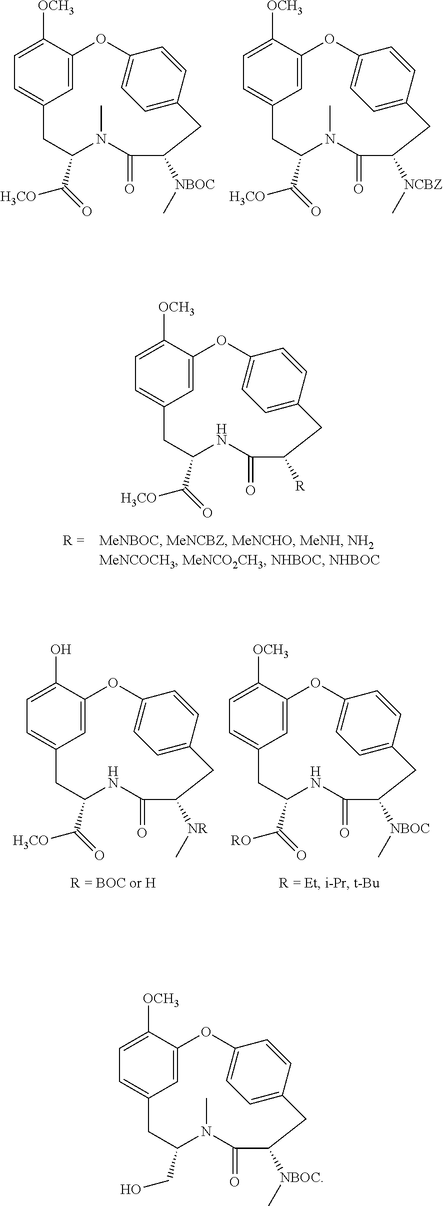

In some embodiments the pharmaceutical composition is:

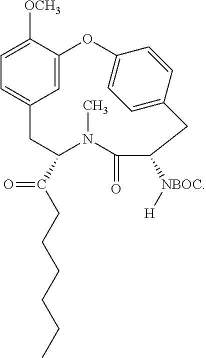

In some embodiments the pharmaceutical composition is:

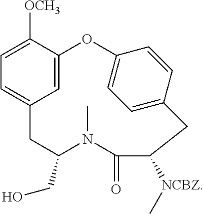

In some embodiments the pharmaceutical composition excludes the structures below:

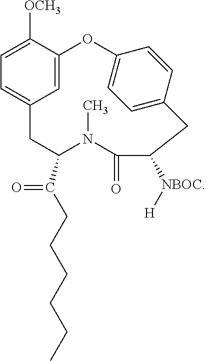

In some embodiments the pharmaceutical composition excludes the structures below:

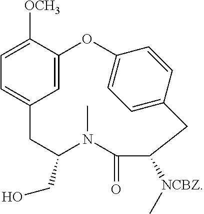

In some embodiments the pharmaceutical composition further comprises an excipient.

In some embodiments the pharmaceutical composition further comprises a binder.

In some embodiments the pharmaceutical composition further comprises a disintegrant.

In some embodiments the pharmaceutical composition inhibits protein translation.

In some embodiments the pharmaceutical composition further comprises a chemotherapeutic agent. In some embodiments the chemotherapeutic agent is paclitaxel.

In some embodiments the invention provides a kit comprising the pharmaceutical composition.

In some embodiments the kit further comprises a chemotherapeutic agent. In some embodiments the chemotherapeutic agent is paclitaxel.

In some embodiments the invention provides a kit comprising an inhibitor of translation and instructions regarding radiation therapy for a human patient in need of radiation treatment. In some aspects the instructions provide a treatment regime. In some embodiments the inhibitor of protein translation is bouvardin or streptovitacin A, or a bouvardin derivative. In some embodiments the inhibitor of protein translation is:

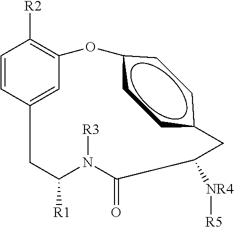

wherein R1 is a CH₂OH or COO-lower alkyl (C1-C6) group, R2 is O-lower alkyl (C1-C6), R3 is H or lower alkyl (C1-C6), R4 is H or a protecting group, and R5 is H or a lower alkyl (C1-C6). In some embodiments the protecting group is BOC or CBZ.

In some embodiments the kit does not include one or more of:

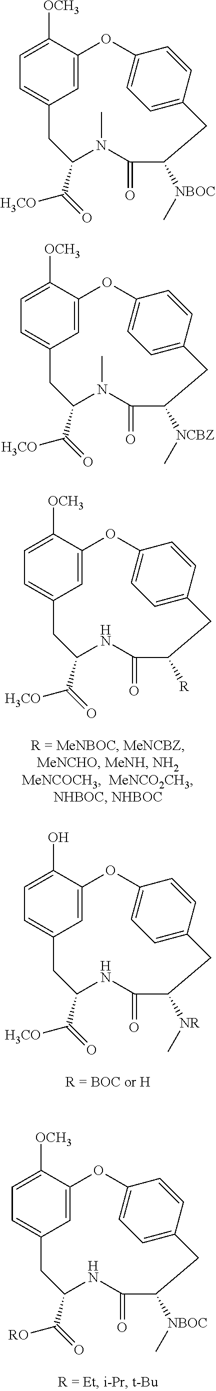

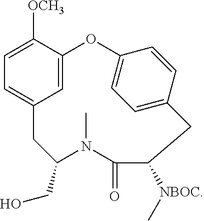

In some embodiments the kit does not include one or more of:

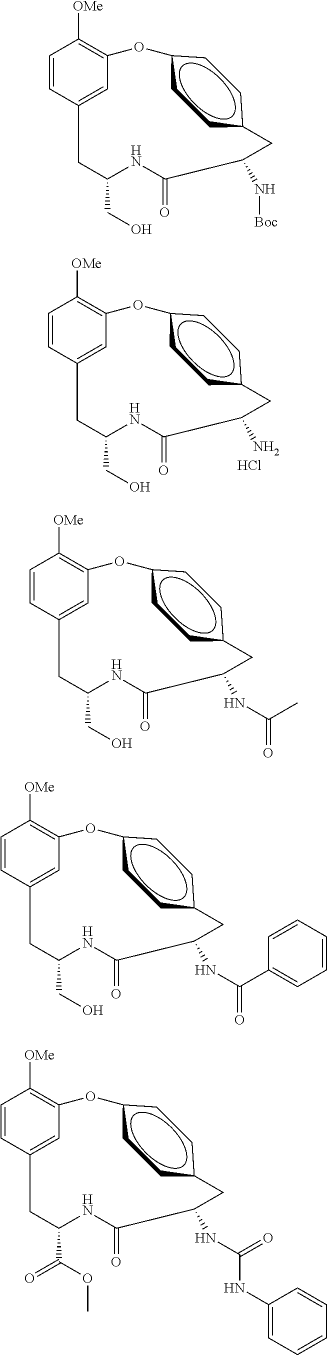

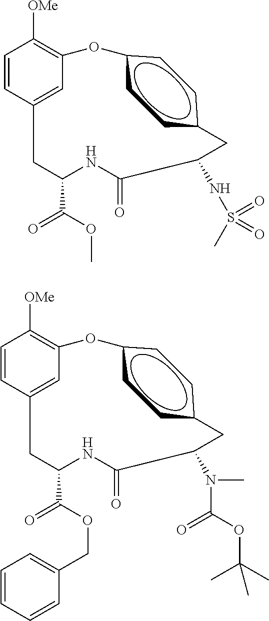

In some embodiments the kit does not include one or more of:

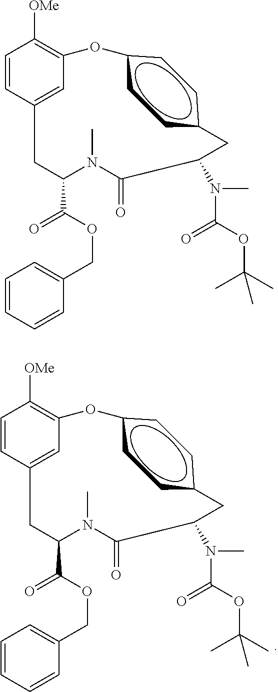

In some embodiments the R1=COOCH₃; R2=OCH₃; R3=H; R4=Boc; R5=CH₃. In some embodiments the R1=CH₂OH, R2=OMe, R3=H, R4=H and R5=H. In some embodiments the R1=CH₂OH, R2=OMe, R3=H, R4=BOC and R5=H.

In some embodiments the kit includes:

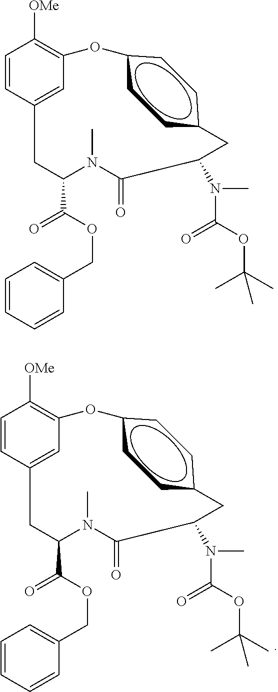

In some embodiments the invention provides for kit comprising an inhibitor of translation and instructions regarding co-treatment of a patient with a chemotherapeutic agent. In some embodiments the chemotherapeutic agent is paclitaxel. In some embodiments the instructions provide a treatment regime. In some embodiments the inhibitor of translation is a compound of the invention.

In some embodiments the invention provides method comprising administering to a patient a composition, wherein a) the patient is in need of treatment, the patient has had radiotherapy, the patient has been diagnosed with cancer, and the patient is a human; and b) the composition comprises a compound of the invention. In some embodiments the compound of the invention is bouvardin. In some embodiments the the amount of bouvardin in the composition would be sub-therapeutic if administered alone. In some embodiments the amount of bouvardin administered is less than 0.01 mg/kg. In some embodiments the amount of bouvardin administered is less than 0.02 mg/kg. In some embodiments the amount of bouvardin administered is less than 0.05 mg/kg. In some embodiments the amount of bouvardin administered is less than 0.1 mg/kg. In some embodiments the amount of bouvardin administered is less than 0.5 mg/kg. In some embodiments the amount of bouvardin administered is less than 1.0 mg/kg. In some embodiments the amount of bouvardin administered is less than 1.5 mg/kg. In some embodiments the amount of bouvardin administered is less than 2.0 mg/kg.

In some embodiments the invention provides method comprising: obtaining a sample from a tumor from a patient with cancer, assessing the level of protein translation said tumor, wherein the level of protein translation can be used to determine whether the patient should be administered a compound of the invention. In some embodiments the patient has been treated with radiotherapy.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts current targets for cancer therapies (arrowheads). Current therapies target every step from growth factor signaling to cell division, with the exception of the ribosome.

FIG. 2 depicts Formula I, bouvardin.

FIG. 3 depicts Formula II, streptovitacin A.

FIG. 4 depicts Formula III, a family of bouvardin derivatives.

FIG. 5 depicts that the chemical entities described herein as “bouvardin derivatives” can be synthesized, e.g., as illustrated with reference to the Reaction Schemes in the figure.

FIG. 6 depicts a rationale for why inhibition of protein synthesis enhances radiation therapy. Radiation kills cells and shrinks tumors but survivors can proliferate to re-populate the tumor. Anti-ribosomals block repopulation.

FIG. 7 depicts that bouvardin (“101” is the compound of Formula I) and a member of the bouvardin derivative family (“101A” is the compound Formula III, with R1=COOCH3, R2=OCH3, R3=H, R4=Boc, R5=CH3) show similar efficacy in Drosophila. Observed effect exceeds additive effect of drug and radiation.

FIG. 8 depicts that bouvardin synergizes with ionizing radiation in H157 lung cancer cells (above) and in Det562 head and neck cancer cells (below).

FIG. 9 depicts that streptovitacin A shows synergystic effect with ionizing radiation (IR) on wild type Drosophila larvae. The observed average for the combination is significantly lower than the expected additive value at 10 μM drug.

FIG. 10 depicts that streptovitacin A alone (without radiation) kills p53 mutant larvae selectively over wild type larvae (SEV) (concentration shown is concentration of streptovitacin A).

FIG. 11 depicts that streptovitacin A synergizes with taxol on Det562 Head and Neck Cancer cells. Concentrations for streptovitacin/taxol are shown. CI=1 indicates additive effects; CI>1 indicates antagonistic effects; CI<1 indicates synergy. The y-axis is “Combination Index” (CI).

FIG. 12 depicts radiation sensitivity of Drosophila larvae in the presence of Bouvardin or Minute mutations.

FIG. 13. depicts that bouvardin inhibits protein translation in vitro.

FIG. 14 depicts CI values for Bouvardin and radiation on 3 cell lines. MTT assays were used to determine the effect of Bouvardin (concentrations in uM) in combination with ionizing radiation (in Gy, X-axis). Combination Index data from 3 different Head and Neck Cancer cell lines are shown. Black dashed lines represent a CI value of 1. CI values below 1 indicate synergy. 1586 and UM-SCC19 were pre-treated whereas HN31 was treated on the same day.

FIG. 15 depicts CI values for Streptovitacin A and radiation on H157 lung cancer cells.

FIG. 16 depicts that bouvardin synergizes with ionizing radiation in human non-small cell lung carcinoma line H157.

FIG. 17 depicts that bouvardin enhances the effect of ionizing radiation on H157 cells in clonogenic assays and xenografts.

FIG. 18 depicts that bouvardin shows synergy in combination with Taxol in FaDu and Det562 head and neck cancer cell lines

FIG. 19 depicts CI values for Bouvardin on FaDu and Det562 head and neck cancer cell lines.

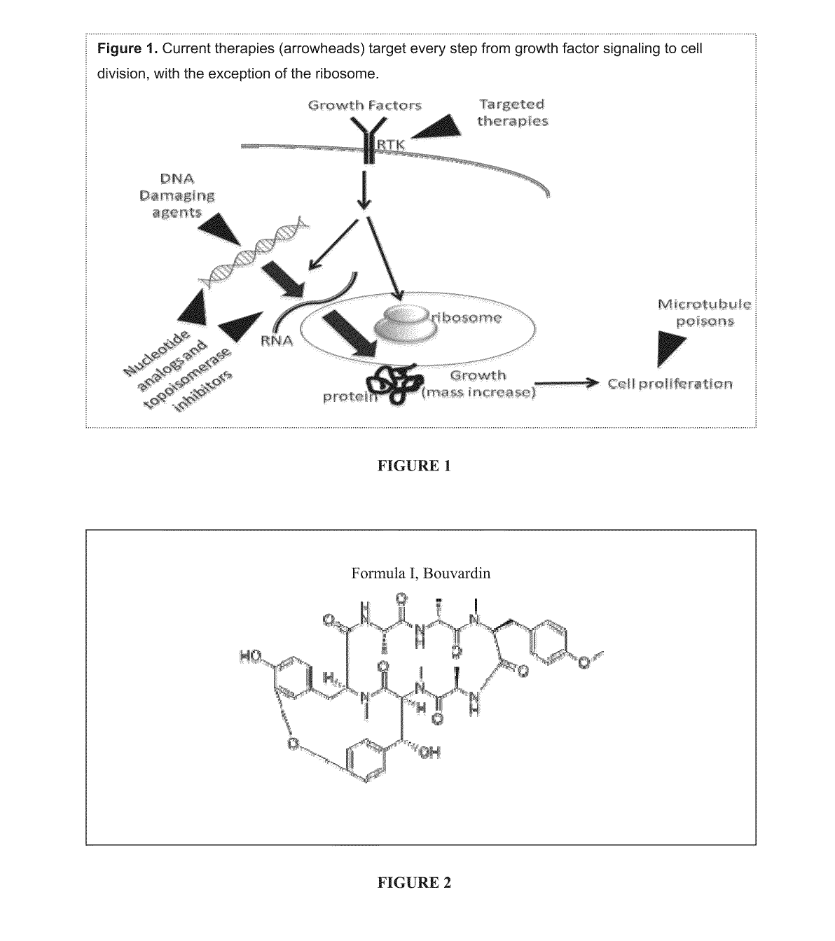

### DETAILED DESCRIPTION OF THE INVENTION

The present invention is directed at agents for the treatment of cancer and diseases associated with proliferation and protein synthesis activity. Provided herein are agents that inhibit ribosome activity, pharmaceutical compositions, and methods for treatments of diseases and conditions associated with ribosome activity or protein accumulation. Compounds of the invention are used alone as therapeutics for cancer and diseases associated with proliferation and protein synthesis activity. Because anti-ribosomals target a previously under-utilized process (translation), compounds of the invention may combine effectively with standard treatments to improve therapy for cancer and diseases associated with proliferation and protein synthesis activity.

### General Considerations

Unless otherwise stated, structures depicted herein are also meant to include compounds that differ by the presence of one or more isotopically enriched atoms. For example, compounds having the present structures except for the replacement of a hydrogen by a deuterium, tritium, or the replacement of a carbon by 13C- or 14C-enriched carbon are within scope of this invention. The compounds of the present invention may also contain unnatural portions of atomic isotopes at one or more of atoms that constitute such compounds. For example, the compounds may be radiolabeled with radioactive isotopes, such as for example tritium, iodine-125, and carbon-14. All isotopic variations of the compounds of the present invention, whether radioactive or not, are encompassed within the scope of the present invention.

When ranges are used herein for physical properties, such as molecular weight, or chemical properties, such as chemical formulae, all combinations and sub combinations of ranges and specific embodiments therein are intended to be included. The term “about” when referring to a number or a numerical range means that the number or numerical range referred to is an approximation within experimental variability (or within statistical experimental error), and thus the number or numerical range may vary between 1% and 15% of the stated number or numerical range.

### DEFINITIONS

The following abbreviation and terms have the indicated meanings throughout.

“Pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions.

“Pharmaceutically acceptable salt” refers to salts that retain the biological effectiveness and properties of the compounds described herein and, which are not biologically or otherwise undesirable. In many cases, the compounds described herein are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

“Solvate” refers to a compound (e.g., a compound selected from Formula I or a pharmaceutically acceptable salt thereof) in physical association with one or more molecules of a pharmaceutically acceptable solvent. It will be understood that “a compound of Formula I” encompasses the compound of Formula I and solvates of the compound, as well as mixtures thereof.

A “therapeutic effect,” as that term is used herein, encompasses a therapeutic benefit and/or a prophylactic benefit. A prophylactic effect includes delaying or eliminating the appearance of a disease or condition, delaying or eliminating the onset of symptoms of a disease or condition, slowing, halting, or reversing the progression of a disease or condition, or any combination thereof.

“Therapeutically effective amount” or “effective amount” refers to that amount of a compound selected from Formula I, Formula II, or Formula III family, that is sufficient to effect a certain action, such as treatment, as defined below, when administered to a mammal in need of such treatment; modulating the catalytic activity of the ribosome, such as when administered to an environment where modulation of the catalytic activity of a ribosome is desired; or disrupting the function of a ribosome, such as when administered to an environment where disrupting the function of a ribosome is desired. The therapeutically effective amount will vary depending upon the subject and disease condition being treated, the weight and age of the subject, the severity of the disease condition, the particular compound selected from Formula I, Formula II, or Formula III family, the dosing regimen to be followed, timing of administration, the manner of administration and the like, all of which can readily be determined by one of ordinary skill in the art.

Where substituent groups are specified by their conventional chemical formulae, written from left to right, they equally encompass the chemically identical substituents that would result from writing the structure from right to left, e.g., —CH₂O— is equivalent to —OCH₂—.

Compounds of Formula I, Formula II or Formula III family also include crystalline and amorphous forms of those compounds, including, for example, polymorphs, pseudopolymorphs, solvates, hydrates, unsolvated polymorphs (including anhydrates), conformational polymorphs, and amorphous forms of the compounds, as well as mixtures thereof. “Crystalline form,” “polymorph,” and “novel form” may be used interchangeably herein, and are meant to include all crystalline and amorphous forms of the compound, including, for example, polymorphs, pseudopolymorphs, solvates, hydrates, unsolvated polymorphs (including anhydrates), conformational polymorphs, and amorphous forms, as well as mixtures thereof, unless a particular crystalline or amorphous form is referred to.

Chemical entities includecompounds of Formula I, Formula II or Formula III family, and all pharmaceutically acceptable forms thereof. Pharmaceutically acceptable forms of the compounds recited herein include pharmaceutically acceptable salts, chelates, non-covalent complexes, prodrugs, and mixtures thereof. In certain embodiments, the compounds described herein are in the form of pharmaceutically acceptable salts. Hence, the terms “chemical entity” and “chemical entities” also encompass pharmaceutically acceptable salts, chelates, non-covalent complexes, prodrugs, and mixtures.

“Pharmaceutically acceptable salts” include, but are not limited to, salts with inorganic acids, such as hydrochlorate, phosphate, diphosphate, hydrobromate, sulfate, sulfinate, nitrate, and like salts; as well as salts with an organic acid, such as malate, maleate, fumarate, tartrate, succinate, citrate, acetate, lactate, methanesulfonate, p-toluenesulfonate, 2-hydroxyethylsulfonate, benzoate, salicylate, stearate, and alkanoate such as acetate, HOOC—(CH₂)ₙ—COON where n ranges from 0 to 4, and like salts. Similarly, pharmaceutically acceptable cations include, but are not limited to sodium, potassium, calcium, aluminum, lithium, and ammonium.

In addition, if a compound is obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare non-toxic pharmaceutically acceptable addition salts.

As noted above, prodrugs also fall within the scope of chemical entities, for example, ester or amide derivatives of the compounds selected from Formula I, Formula II or Formula III. The term “prodrug” includes any compound that becomes a compound of Formula I or Formula II when administered to a patient, e.g., upon metabolic processing of the prodrug. Examples of prodrugs include, but are not limited to, acetate, formate, benzoate, and like derivatives of functional groups (such as alcohol or amine groups) in the compounds selected from Formula I, Formula II or Formula III family.

The term “chelate” refers to the chemical entity formed by the coordination of a compound to a metal ion at two (or more) points.

The term “non-covalent complex” refers to the chemical entity formed by the interaction of a compound and another molecule wherein a covalent bond is not formed between the compound and the molecule. For example, complexation can occur through van der Waals interactions, hydrogen bonding, and electrostatic interactions (also called ionic bonding).

The term “active agent” is used to indicate a chemical entity which has biological activity. In certain embodiments, an “active agent” is a compound having pharmaceutical utility.

“Patient” refers to an animal, such as a mammal, for example a human that has been or will be the object of treatment, observation or experiment. The methods described herein can be useful in both human therapy and veterinary applications. In some embodiments, the patient is a mammal, and in some embodiments, the patient is human.

“Treatment” or “treating” means any treatment of a disease in a patient, including: preventing the disease, that is, causing the clinical symptoms of the disease not to develop; inhibiting the disease; slowing or arresting the development of clinical symptoms; and/or relieving the disease, that is, causing the regression of clinical symptoms.

The term “selective inhibition” or “selectively inhibit” as referred to a biologically active agent refers to the agent's ability to preferentially reduce the target signaling activity as compared to off-target signaling activity, via direct or interact interaction with the target.

“Combination Index” computation. Combination Indices were calculated using CalcuSyn software (Biosoft), which uses the method of Chou and Talalay. This method first computes drug-induced effect using the Hill Equation: where E is the measured effect; C is the drug concentration; Emax is the full range of drug effect, usually at or near 100%; IC50 is the drug concentration producing the median effect of 50%; and n is the curve shape parameter describing the steepness of the concentration-effect relationship. The Chou and Talalay method then linearizes the Hill Equation by logarithmic transformation as: where fu is the fraction of cells left unaffected after drug exposure, fa is the fraction of cells affected by the exposure, C is the drug concentration used, Cm is the concentration to achieve the median effect, and n is the curve shape parameter. Cm and n are equivalent to IC50 and n, respectively, in the Hill Equation. The values of n (obtained from the slope), nlog(Cm) (obtained from the absolute value of the intercept), and, therefore, Cm are obtained by plotting log(fu−1−1) versus log(C). These numbers are then used to compute the Combination Index (CI) according to the formula: CI=(D)1/(Dx)1+(D)2/(Dx)2 (26). (Dx)1 and (Dx)2 are the doses required to achieve a given effect level for each treatment, i.e. a specified value of Fraction Affected or Fa. (D)1 and (D)2 are the doses of each treatment in a given combination which gives the same Fa.

“Standard deviation” Standard deviation for expected fraction survival for co-treatment of drug and radiation is computed according to the formula for the standard deviation of a product of two normally distributed variables. For two normally distributed variables with means m1 and m2 and standard deviations s1 and s2, the product will have mean m1 m2 and the standard deviation=square root of (m12s22+m22s12+s12s22).

### Compounds of the Invention

Compounds of the invention are provided herein. Such compounds include “bouvardin” “streptovitacin A” and “bouvardin derivatives.”

In one aspect, the invention provides the compound “bouvardin” and its pharmaceutically acceptable salts. FIG. 2 illustrates bouvardin, the compound of formula (I):

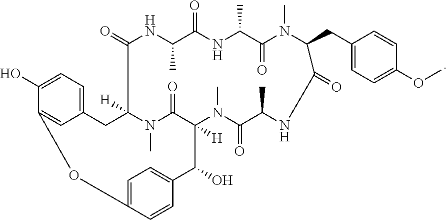 [SMILES: COc1ccc(C[C@H]2C(=O)N[C@H](C)C(=O)N(C)[C@@H]3C(=O)N(C)[C@@H](Cc4ccc(O)c(c4)Oc4ccc(cc4)[C@H]3O)C(=O)N[C@@H](C)C(=O)N[C@H](C)C(=O)N2C)cc1]

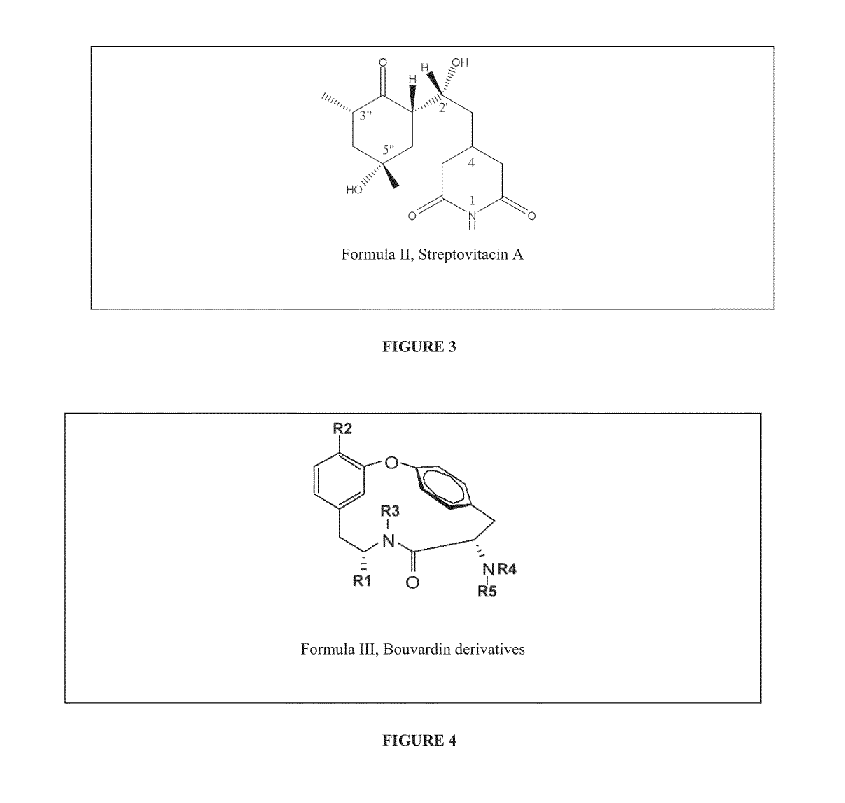

In one aspect, the invention provides compound “streptovitacin A” and its pharmaceutically acceptable salts. FIG. 3 illustrates “streptovitacin A,” the compound of formula (II):

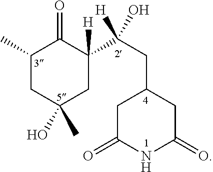 [SMILES: C[C@H]1C[C@@](C)(O)C[C@@H]([C@H](O)CC2CC(=O)NC(=O)C2)C1=O]

In one aspect, the invention provides compound family of “derivatives of bouvardin,” and pharmaceutically acceptable salts thereof. FIG. 4 illustrates the general structure of derivatives of bouvardin, which differ from bouvardin in that they lack a cyclic hexapeptide group, as a general formula (III):

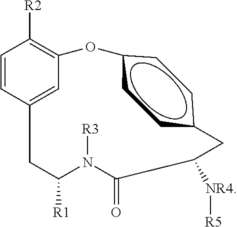

In one embodiment, a derivative of bouvardin has the formula depicted in formula III, wherein R1=COOCH₃; R2=OCH₃; R3=H; R4=Boc (Boc=tert-Butyloxycarbonyl, herein also “BOC”); R5=CH₃.

In one embodiment, a derivative of bouvardin has the formula depicted in formula III, wherein R1=COOCH₃; R2=OCH₃; R3=H; R4=H; R5=CH₃.

In some embodiments, a family of derivatives of bouvardin has the formula depicted in formula III wherein R1 is a CH₂OH or COO-lower alkyl (C1-C6) group, R2 is O-lower alkyl (C1-C6), R3 is H or lower alkyl (C1-C6), R4 is H or a protecting group, such as BOC or CBZ (carbobenzyloxy), and R5 is H or a lower alkyl (C1-C6).

In some embodiments the derivative of bouvardin is or includes one or more of the following:

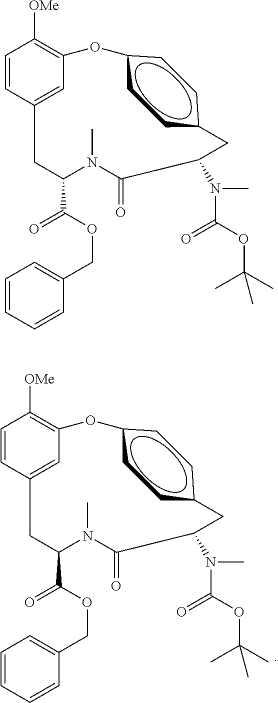

In some embodiments the derivatives of bouvardin include the stereoisomers at R1 of the formula depicted in formula III.

In one embodiment, a derivative of bouvardin does not have the formula depicted in formula III, wherein R1=COOCH₃; R2=OCH₃; R3=H; R4=Boc (Boc=tert-Butyloxycarbonyl, herein also “BOC”); R5=CH₃.

In one embodiment, a derivative of bouvardin does not have the formula depicted in formula III, wherein R1=COOCH₃; R2=OCH₃; R3=H; R4=H; R5=CH₃.

In some embodiments the derivative of bouvardin is not one or more of the following:

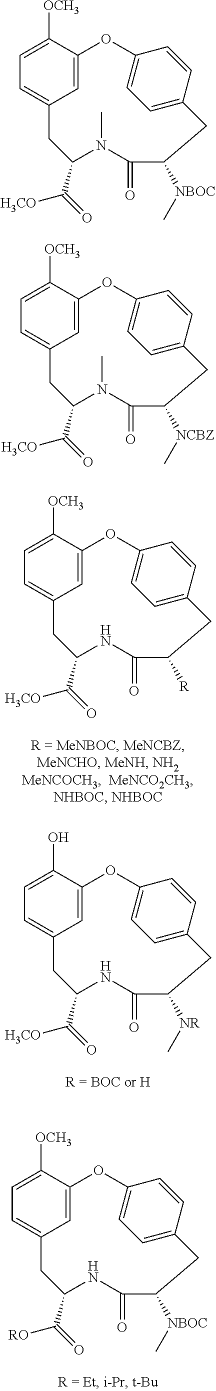

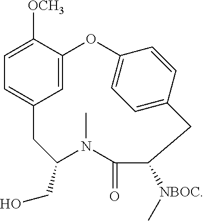

In some embodiments the derivative of bouvardin has the formula depicted in Formula III, wherein R1 is not COOCH₃; R2 is not OCH₃; R3 is not H; R4 is not Boc (Boc=tert-Butyloxycarbonyl, herein also “BOC”); R5=CH₃.

In some embodiments the derivative of bouvardin is not one or more of the following:

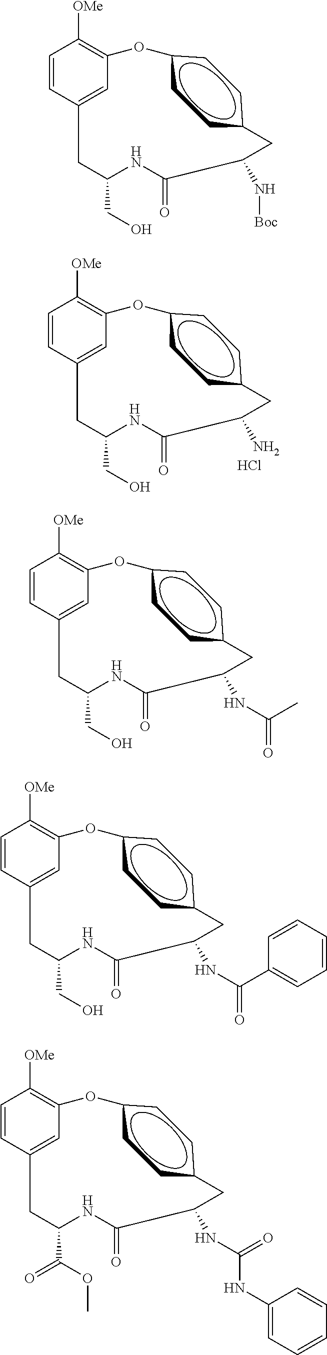

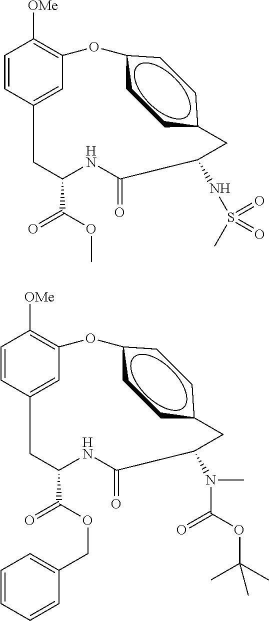

In some embodiments the derivative of bouvardin is not one or more of the following:

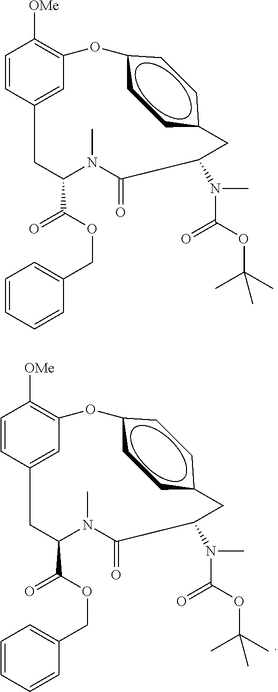

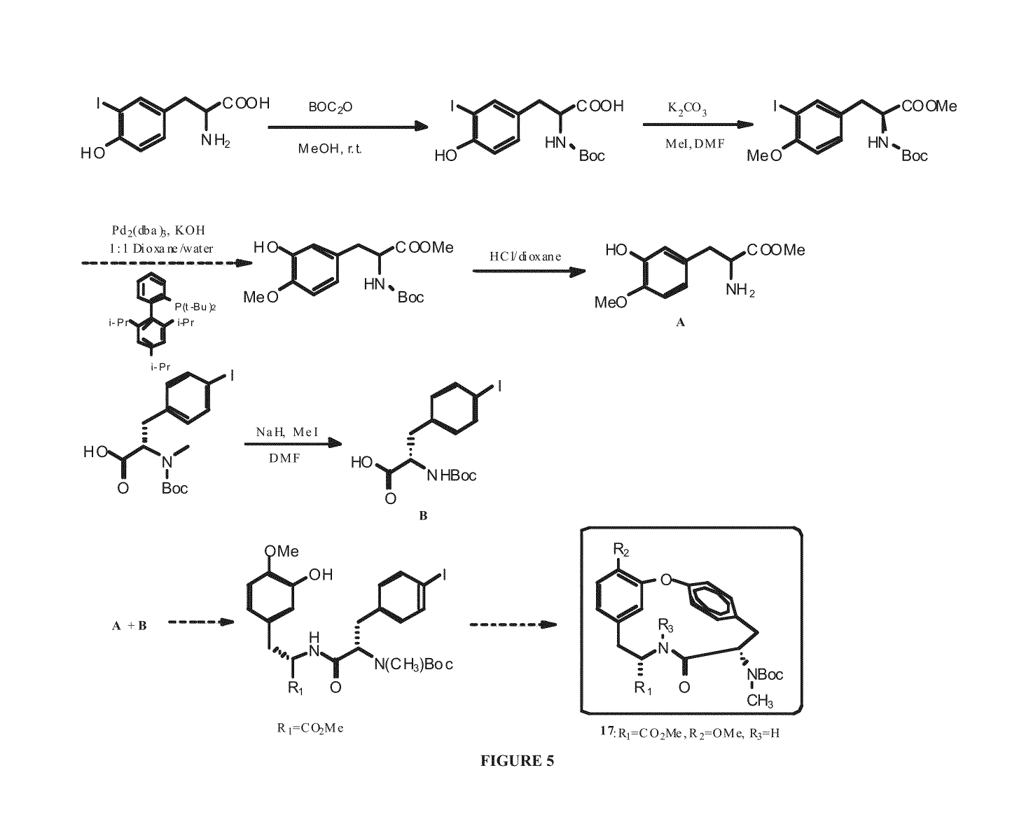

The chemical entities described herein as “bouvardin derivatives” can be synthesized utilizing techniques well known in the art, e.g., as illustrated in FIG. 5 with reference to the Reaction Schemes in that Figure. The bouvardin derivatives with the a COO-lower alkyl group at the R1 position can be converted to derivatives with a CH₂OH group at the R1 position via selective reduction techniques, which are well known in the art.

Unlike the synthesis of bouvardin, which took about 30 steps to complete (see Dale L. Boger, Michael A. Patane, Jiacheng Zhou J. Am. Chem. Soc., 1994, 116 (19), pp. 8544-8556), synthesis of a representative compound in the less structurally complex bouvardin derivative family, which lacks the hexapeptide ring, takes only seven steps (see FIG. 5). Nevertheless, compounds in the bouvardin derivative family have been shown to be as effective as bouvardin itself in sensitizing flies to ionizing radiation, which is commonly used for radiation therapy in human cancers (see Examples). Therefore, bouvardin derivatives of the present invention may be as effective as the complex parent bouvardin structure for carrying out treatment methods described herein, and the use of bouvardin derivatives is highly advantageous over the use of bouvardin itself due to significant cost and time savings associates the relatively facile synthesis of bouvardin derivatives.

Unless specified to the contrary, the reactions described herein take place at atmospheric pressure, generally within a temperature range from −10° C. to 200° C. Further, except as otherwise specified, reaction times and conditions are intended to be approximate, e.g., taking place at about atmospheric pressure within a temperature range of about −10° C. to about 110° C. over a period of about 1 to about 24 hours. The terms “solvent,” “organic solvent,” and “inert solvent” each mean a solvent inert under the conditions of the reaction being described in conjunction therewith including, for example, benzene, toluene, acetonitrile, tetrahydrofuran (“THF”), dimethylformamide (“DMF”), chloroform, methylene chloride (or dichloromethane), diethyl ether, methanol, N-methylpyrrolidone (“NMP”), pyridine and the like. Unless specified to the contrary, the solvents used in the reactions described herein are inert organic solvents. Unless specified to the contrary, for each gram of the limiting reagent, one cc (or mL) of solvent constitutes a volume equivalent.

Isolation and purification of the chemical entities and intermediates described herein can be effected, if desired, by any suitable separation or purification procedure such as, for example, filtration, extraction, crystallization, column chromatography, thin-layer chromatography or thick-layer chromatography, or a combination of these procedures. Specific illustrations of suitable separation and isolation procedures can be had by reference to the examples herein below. However, other equivalent separation or isolation procedures can also be used.

When desired, the (R)- and (S)-isomers may be resolved by methods known to those skilled in the art, for example by formation of diastereoisomeric salts or complexes which may be separated, for example, by crystallization; via formation of diastereoisomeric derivatives which maybe separated, for example, by crystallization, gas-liquid or liquid chromatography; selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. Alternatively, a specific enantiomer may be synthesized by asymmetric synthesis using optically active reagents, substrates, catalysts or solvents, or by converting one enantiomer to the other by asymmetric transformation.

The chemical entities can be synthesized by an appropriate combination of generally well-known synthetic methods. Techniques useful in synthesizing the chemical entities are both readily apparent and accessible to those of skill in the relevant art. A racemic mixture can be optionally placed on a chromatography column and separated into (R)- and (S)-enantiomers. The compounds described herein can be optionally contacted with a pharmaceutically acceptable acid to form the corresponding acid addition salts.

In some aspects, the invention provides a composition that contains a compound of the present invention, In some aspects, the concentration of one or more of the compounds is less than 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, 0.0001% w/w or v/v.

In some aspects, the concentration of one or more of the compounds of the present invention is greater than 90%, 80%, 70%, 60%, 50%, 40%, 30%, 20%, 19.75%, 19.50%, 19.25%, 19%, 18.75%, 18.50%, 18.25%, 18%, 17.75%, 17.50%, 17.25%, 17%, 16.75%, 16.50%, 16.25%, 16%, 15.75%, 15.50%, 15.25%, 15%, 14.75%, 14.50%, 14.25%, 14%, 13.75%, 13.50%, 13.25%, 13%, 12.75%, 12.50%, 12.25%, 12%, 11.75%, 11.50%, 11.25%, 11%, 10.75%, 10.50%, 10.25%, 10%, 9.75%, 9.50%, 9.25%, 9%, 8.75%, 8.50%, 8.25%, 8%, 7.75%, 7.50%, 7.25%, 7%, 6.75%, 6.50%, 6.25%, 6%, 5.75%, 5.50%, 5.25%, 5%, 4.75%, 4.50%, 4.25%, 4%, 3.75%, 3.50%, 3.25%, 3%, 2.75%, 2.50%, 2.25%, 2%, 1.75%, 1.50%, 1.25%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002%, 0.0001% w/w or v/v.

In some aspects, the concentration of one or more of the compounds of the present invention is in the range from approximately 0.0001% to approximately 50%, approximately 0.001% to approximately 40%, approximately 0.01% to approximately 30%, approximately 0.02% to approximately 29%, approximately 0.03% to approximately 28%, approximately 0.04% to approximately 27%, approximately 0.05% to approximately 26%, approximately 0.06% to approximately 25%, approximately 0.07% to approximately 24%, approximately 0.08% to approximately 23%, approximately 0.09% to approximately 22%, approximately 0.1% to approximately 21%, approximately 0.2% to approximately 20%, approximately 0.3% to approximately 19%, approximately 0.4% to approximately 18%, approximately 0.5% to approximately 17%, approximately 0.6% to approximately 16%, approximately 0.7% to approximately 15%, approximately 0.8% to approximately 14%, approximately 0.9% to approximately 12%, approximately 1% to approximately 10% w/w, w/v, or v/v.

In some aspects, the concentration of one or more of the compounds of the present invention is in the range from approximately 0.001% to approximately 10%, approximately 0.01% to approximately 5%, approximately 0.02% to approximately 4.5%, approximately 0.03% to approximately 4%, approximately 0.04% to approximately 3.5%, approximately 0.05% to approximately 3%, approximately 0.06% to approximately 2.5%, approximately 0.07% to approximately 2%, approximately 0.08% to approximately 1.5%, approximately 0.09% to approximately 1%, approximately 0.1% to approximately 0.9% w/w. w/v, or v/v.

In some aspects, the amount of one or more of the compounds of the present invention administered to a subject is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g. 0.0008 g, 0.0007 g, 0.0006 g, 0.0005 g, 0.0004 g, 0.0003 g, 0.0002 g, or 0.0001 g.

In some aspects, the amount of one or more of the compounds of the present invention administered to a subject is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5 g, 3 g, 3.5 g, 4 g, 4.5 g, 5 g, 5.5 g, 6 g, 6.5 g, 7 g, 7.5 g, 8 g, 8.5 g, 9 g, 9.5 g, or 10 g.

In some aspects the amount of one or more of the compounds of the present invention administered to a subject is in the range of 0.0001 g to 10 g, 0.0005 g to 9 g, 0.001 g to 8 g, 0.005 g to 7 g, 0.01 g to 6 g, 0.05 g to 5 g, 0.1 g to 4 g, 0.5 g to 4 g, or 1 g to 3 g.

In some embodiments the combination of radiation therapy will allow for a lower useful does of a compound of the invention. In some embodiments less bouvardin is used in combination with radiation therapy. For example, in some embodiments less than 0.01 mg/kg, less than 0.02 mg/kg, less than 0.05 mg/kg, less than 0.1 mg/kg, less than 0.5, less than 1.0 mg/kg, less than 1.5 mg/kg, or less than 2.0 mg/kg of bouvardin can be used. In some embodiments less than 0.01 mg/kg, less than 0.02 mg/kg, less than 0.05 mg/kg, less than 0.1 mg/kg, less than 0.5, less than 1.0 mg/kg, less than 1.5 mg/kg, or less than 2.0 mg/kg of a bouvardin derivative can be used.

In some embodiments more than on bouvardin derivatives is used in combination or in a single composition.

The compounds according to the invention are effective over a wide dosage range. The exact dosage will depend upon the route of administration, the form in which the compound is administered, the subject to be treated, the body weight of the subject to be treated, and the preference and experience of the attending physician.

The compounds of the present invention are usually administered in the form of pharmaceutical compositions. The other agents described herein are also administered in the form of pharmaceutical compositions. When the compounds of the present invention are used in combination with other agents, both components may be mixed into a preparation or both components may be formulated into separate preparations to use them in combination separately or at the same time.

In some aspects this invention therefore provides pharmaceutical compositions that contain, as the active ingredient, a compound of the present invention or a pharmaceutically acceptable salt and/or coordination complex thereof, a second agent or a pharmaceutically acceptable salt and/or coordination complex thereof, and one or more pharmaceutically acceptable, excipients, carriers, include including inert solid diluents and fillers, diluents, including sterile aqueous solution and various organic solvents, permeation enhancers, solubilizers, and adjuvants.

The compound of the present invention may be prepared into pharmaceutical compositions in dosages as described herein see (e.g., “Pharmaceutical compositions for oral administration”). Such compositions are prepared by methods that are well known in the pharmaceutical arts.

### Pharmaceutical Compositions for Oral Administration.

In some aspects, the invention provides a pharmaceutical composition for oral administration containing a compound of the present invention, and a pharmaceutical excipient suitable for oral administration.

In some aspects, the invention provides a solid pharmaceutical composition for oral administration containing: (i) an effective amount of a compound of the present invention; (ii) an effective amount of a second agent; and (iii) a pharmaceutical excipient suitable for oral administration. In some embodiments, the composition further contains: (iv) an effective amount of a third agent.

In some aspects, the pharmaceutical composition may be a liquid pharmaceutical composition suitable for oral consumption. Pharmaceutical compositions of the invention suitable for oral administration can be presented as discrete dosage forms such as capsules, cachets, tablets, or liquids, or aerosol sprays each containing a predetermined amount of an active ingredient as a powder or in granules, a solution, or a suspension in an aqueous or non-aqueous liquid, an oil-in-water emulsion, or a water-in-oil liquid emulsion. Such dosage forms can be prepared by any of the methods of pharmacy, but all methods include the step of bringing the active ingredient into association with the carrier, which constitutes one or more necessary ingredients. In general, the compositions are prepared by uniformly and intimately admixing the active ingredient with liquid carriers or finely divided solid carriers or both, and then, if necessary, shaping the product into the desired presentation. For example, a tablet can be prepared by compression or molding, optionally with one or more necessary ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surface active or dispersing agent. Molded tablets can be made by molding in a suitable machine a mixture of the powdered compound moistened with an inert liquid diluent.

In some aspects, this invention further encompasses anhydrous pharmaceutical compositions and dosage forms comprising an active ingredient, since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms of the present invention can be prepared using anhydrous or low moisture containing ingredients and low moisture or low humidity conditions. Pharmaceutical compositions and dosage forms of the invention which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs and strip packs.

In some aspects, an active ingredient is combined in an intimate admixture with a pharmaceutical carrier according to conventional pharmaceutical compounding techniques. In various aspects the carrier takes a wide variety of forms depending on the form of preparation desired for administration. In preparing the compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents, can be used in the case of oral solid preparations, in some embodiments without employing the use of lactose. For example, suitable carriers include powders, capsules and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxylmethyl cellulose), polyvinyl pyrrolidone, methyl cellulose, pre-gelatinized starch, hydroxypropyl methyl cellulose, microcrystalline cellulose, and mixtures thereof.

Examples of suitable fillers for use in the pharmaceutical compositions and dosage forms disclosed herein include, but are not limited to, talc, calcium carbonate (e.g., granules or powder) microcrystalline cellulose, powdered cellulose, dextrates, kaolin, mannitol, silicic acid, sorbitol, starch, pre-gelatinized starch, and mixtures thereof.

Disintegrants may be used in the composition of the invention to provide tablets that disintegrated when exposed to an aqueous environment. Too much of a disintegrant may produce tables which may disintegrate in the bottle. Too little may be insufficient for disintegration to occur and may thus alter the rate and extent of release of the active ingredient(s) from the dosage form. Thus, a sufficient amount of disintegrant that is neither too little nor too much to detrimentally alter the release of the active ingredient(s) may be used to form the dosage forms of the compounds disclosed herein. The amount of disintegrant used may vary based upon the type of formulation and mode of administration, and may be readily discernible to those of ordinary skill in the art. About 0.5 to about 15 weight percent of disintegrant or about 1 to about 5 weight percent of disintegrant, may be used in the pharmaceutical composition. Disintegrants thatn can be used to form pharmaceutical compositions and dosage forms of the invention include, but are not limited to, agar-agar, alginic acid, calcium carbonate, microcrystalline cellulose, crocarmellose sodium, crospovidone, polacrilin potassium, sodium starch glycolate, potato or tapioca starch, other starches, pre-gelatanized starch, other starches, clays, other algins, other cellulose, gums or mixtures thereof.

Lubricants which can be used to form pharmaceutical compostions and dosage forms of the invention include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, a syloid silica gel, a coagulated aerosol of synthetic silica, or mixtures thereof. A lubricant can be optionally added, in an amount of less than about 1 weight percent of the pharmaceutical composition.

When aqueous suspensions and/or elixirs are desired for oral administration, theactive ingredient therein may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate, or kaolin, or as soft gelatin capsules wherein the active ingredient is mixed with water or an oil medium, for example, peanut oil, liquid paraffin, or olive oil.

Surfactant which can be used to form pharmaceutical compositions and dosage forms of the invention include, but are not limited to, hydrophilic surfactants, lipophilic surfactants, and mixtures thereof. That is, a mixture of hydrophilic surfactants may be employed, or a mixture of at least one hydrophilic surfactant and at least one lipophilic surfactant may be employed.

A suitable hydrophilic surfactant may generally have an HLB value of at least 10, while lipophilic surfactants may generally have an HLB value of or less than about 10. An empirical parameter is used to characterize the relative hydrophilicity and hydrophobicity of non-ionic amphiphilic compounds is the hydrophilic-lipophilic balance (“HLB value”). Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e. hydrophobic) surfactants are compounds having an HLB value equal to or less than about 10. However, HLB value of a surfactant is merely a rough guide generally used to enable formulation of industrial pharmaceutical and cosmetic emulsions.

Hydrophilic surfactants may be wither ionic or non-ionic. Suitable ionic surfactants include, but are not limited to, alklylammonium salts; fusidic acid salts, fatty acid salts; fatty acid derivatives of amino acids, oligopeptides and polypeptides; glyceride derivatives of amino acids, oligopeptides and polypeptides; lecithins and hydrogenated lecithins; lysolecithins and hydrogenated lethicithins; phospholipids and derivatives thereof; lysophospholipids and derivatives thereof; carnitine fatty acid ester salts; salts of alkylsulfates; fatty acid salts; sodium docusate; acyltactylates; mono- and di-acetylated tartaric acid esters of mono- and di-glycerides; succinylated mono- and di-glycerides; citric acid esters of mono- and di-glycerides; and mixtures thereof.

Within the aforementioned group, preferred ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinlylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phoshatidic acid, phosphatidylserine, lysophosphatidylcholine, lysophosphatidylethanolamine, lysophosphatidylglycerol, lysophosphatidic acid, lysophosphatidylerine, PEG-phosphatidylethanolamine, PVP-phosphatidylethanolamine, lactylic esters of fatty acids, stearoyl-2-lactylate, succinylated mono glycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carntines, palmitoyl carnitines, myristoyl carnitines, and salts and mixtures thereof.

Hydrophilic non-ionic surfactants may include, but not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides. In one embodiment, the composition may include a solubilizer to ensure good solubilization and/or dissolution of the compound of the present invention and to minimize precipitation of the compound of the present invention. This can be especially important for compositions for non-oral use, e.g., compositions for injection. A solubilizer may also be added to increase the solubility of the hydrophilic drug and/or other components, such as surfactants, or to maintain the composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, δ-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water. Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the composition to a patient using conventional techniques, such as distillation or evaporation. Thus, if present, the solubilizer can be in a weight ratio of 10%, 25%, 50%, 100%, or up to about 200% by weight, based on the combined weight of the drug, and other excipients. If desired, very small amounts of solubilizer may also be used, such as 5%, 2%, 1% or even less. Typically, the solubilizer maybe present in an amount of about 1% to about 100%, more typically about 5% to about 25% by weight.

The composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

In addition, an acid or a base may be incorporated into the composition to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like.

### Pharmaceutical Compositions for Injection.

In some embodiments, the invention provides a pharmaceutical composition for injection containing a compound of the present invention and a pharmaceutical excipient suitable for injection. Components and amounts of agents in the compositions are as described herein.

The forms in which the novel compositions of the present invention maybe incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the compound of the present invention in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Pharmaceutical Compositions for Topical and/or Transdermal Delivery.

In some embodiments, the invention provides a pharmaceutical composition for topical and/or transdermal delivery containing a compound of the present invention and a pharmaceutical excipient suitable for topical and/or transdermal delivery.

Compositions of the present invention can be formulated into preparations in solid, semi-solid, or liquid forms suitable for local or topical administration, such as gels, water soluble jellies, creams, lotions, suspensions, foams, powders, slurries, ointments, solutions, oils, pastes, suppositories, sprays, emulsions, saline solutions, dimethylsulfoxide (DMSO)-based solutions. In general, carriers with higher densities are capable of providing an area with a prolonged exposure to the active ingredients. In contrast, a solution formulation may provide more immediate exposure of the active ingredient to the chosen area.

The pharmaceutical compositions also may comprise suitable solid or gel phase carriers or excipients, which are compounds that allow increased penetration of, or assist in the delivery of, therapeutic molecules across the stratum corneum permeability barrier of the skin. There are many of these penetration-enhancing molecules known to those trained in the art of topical formulation. Examples of such carriers and excipients include, but are not limited to, humectants (e.g., urea), glycols (e.g., propylene glycol), alcohols (e.g., ethanol), fatty acids (e.g., oleic acid), surfactants (e.g., isopropyl myristate and sodium lauryl sulfate), pyrrolidones, glycerol monolaurate, sulfoxides, terpenes (e.g., menthol), amines, amides, alkanes, alkanols, water, calcium carbonate, calcium phosphate, various sugars, starches, cellulose derivatives, gelatin, and polymers such as polyethylene glycols.

Another preferred formulation for use in the methods of the present invention employs transdermal delivery devices (“patches”). Such transdermal patches may be used to provide continuous or discontinuous infusion of a compound of the present invention in controlled amounts, either with or without another agent.

The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

### Pharmaceutical Compositions for Inhalation.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a face mask tent, or intermittent positive pressure breathing machine. Solution, suspension, or powder compositions may be administered, preferably orally or nasally, from devices that deliver the formulation in an appropriate manner

### Other Pharmaceutical Compositions.

Pharmaceutical compositions may also be prepared from compositions described herein and one or more pharmaceutically acceptable excipients suitable for sublingual, buccal, rectal, intraosseous, intraocular, intranasal, epidural, or intraspinal administration. Preparations for such pharmaceutical compositions are well-known in the art. See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, N.Y., 1990; Katzung, ed., Basic and Clinical Pharmacology, Ninth Edition, McGraw Hill, 2004; Goodman and Gilman, eds., The Pharmacological Basis of Therapeutics, Tenth Edition, McGraw Hill, 2001; Remingtons Pharmaceutical Sciences, 20th Ed., Lippincott Williams &Wilkins., 2000; Martindale, The Extra Pharmacopoeia, Thirty-Second Edition (The Pharmaceutical Press, London, 1999); all of which are incorporated by reference herein in their entirety.

The invention also provides kits. The kits include a compound or compounds of the present invention as described herein, in suitable packaging, and written material that can include instructions for use, discussion of clinical studies, listing of side effects, and the like. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. The kit may further contain another agent. In some embodiments, the compound of the present invention and the agent are provided as separate compositions in separate containers within the kit. In some embodiments, the compound of the present invention and the agent are provided as a single composition within a container in the kit. Suitable packaging and additional articles for use (e.g., measuring cup for liquid preparations, foil wrapping to minimize exposure to air, and the like) are known in the art and may be included in the kit. Kits described herein can be provided, marketed and/or promoted to health providers, including physicians, nurses, pharmacists, formulary officials, and the like. Kits may also, in some embodiments, be marketed directly to the consumer.

### Additional Illustrative Compounds of the Invention Include the Following Embodiments:

The invention provides a pharmaceutical composition comprising one or more compounds disclosed herein. In some embodiments the invention provides pharmaceutical compositions for the treatment of cancer and diseases associated with proliferation and protein synthesis activity in a mammal. In some embodiment, the treatment of said disorders comprises a therapeutically effective amount of a compound of the present invention, or a pharmaceutically acceptable salt, ester, prodrug, solvate, hydrate, or derivative thereof, and a pharmaceutically acceptable carrier.

In some aspects the compositions or compounds of the invention relate to the treatment of cancer, such as acute myeloid leukemia, thymus, brain, lung, squamous cell, skin, eye, retinoblastoma, intraocular melanoma, oral cavity and oropharyngeal, bladder, gastric, stomach, pancreatic, breast, cervical, head, neck, renal, kidney, liver, ovarian, prostate, colorectal, esophageal, testicular, gynecological, thyroid, CNS, PNS, AIDS-related (e.g. Lymphoma and Kaposi's Sarcoma), or Viral-Induced cancer. In some embodiments, the pharmaceutical composition is for the treatment of a non-cancerous hyper proliferative disorder such as benign hyperplasia of the skin (e.g., psoriasis), restinosis, or prostate (e.g., benign prostactic hypertrophy (BPH)).

In some aspects the compositions or compounds of the invention relate to the treatment of diabetes in a mammal

In some embodiments the invention also relates to compositions for the treatment of pancreatitis or kidney disease (including proliferative glomerulonephritis and diabetes-induced renal disease) or pain in a mammal which comprises a therapeutically effective amount of a compound of the present invention, or a pharmaceutically acceptable salt, ester, prodrug, solvate, hydrate, or derivative thereof, and a pharmaceutically acceptable carrier.

In some embodiments the invention also relates to a composition for treating a disease related to vasculogenesis or angiogenesis in a mammal. In some embodiments, the invention relates to pharmaceutical compositions for treating a disease related to vasculogenesis or angiogenesis in a mammal which comprises a therapeutically effective amount of a compound of the present invention, or a pharmaceutically acceptable salt, ester, prodrug, solvate, hydrate, or derivative thereof, and a pharmaceutically acceptable carrier. In some embodiments, said pharmaceutical composition is for treating a disease selected from the group consisting of tumor angiogenesis, chronic inflammatory disease such as rheumatoid arthritis, inflammatory bowel disease, atherosclerosis, skin diseases such as psoriasis, eczema, and scleroderma, diabetes, diabetic retinopathy, sarcoma and ovarian, breast, lung, pancreatic, prostate, colon, and epidermoid cancer.

In some aspects the LD50 in mice of compounds disclosed herein are within the range shown by FDA-approved chemotherapy agents, such as 0.01 mg/kg to 10,000 mg/kg, or 0.1 mg/kg to 1,000 mg/kg, or 1 mg/kg to 100 mg/kg. In some embodiments, the LD50 of bouvardin is 12.4 mg/kg.

### Methods

In some aspects the invention provides a method of treating a hyperproliferative disorder in a mammal that comprises administering to the mammal a therapeutically effective amount of a compound of the present invention, or a pharmaceutically acceptable salt, ester, prodrug, solvate, hydrate or derivative thereof. In some embodiments, said method relates to the treatment of cancer such as acute myeloid leukemia, thymus, brain, lung, squamous cell, skin, eye, retinoblastoma, intraocular melanoma, oral cavity and oropharyngeal, bladder, gastric, stomach, pancreatic, bladder, breast, cervical, head, neck, renal, kidney, liver, ovarian, prostate, colorectal, esophageal, testicular, gynecological, thyroid, CNS, PNS, AIDS-related (e.g. Lymphoma and Kaposi's Sarcoma) or viral-induced cancer. In some embodiments, said method relates to the treatment of a non-cancerous hyperproliferative disorder such as benign hyperplasia of the skin (e. g., psoriasis), restenosis, or prostate (e. g., benign prostatic hypertrophy (BPH)).

In some aspects the invention provides a method for the treatment of a hyperproliferative disorder in a mammal that comprises administering to the mammal a therapeutically effective amount of a compound of the present invention, or a pharmaceutically acceptable salt, ester, prodrug, solvate, hydrate or derivative thereof, in combination with an anti-tumor agent. In some embodiments, the anti-tumor agent is selected from the group consisting of mitotic inhibitors, alkylating agents, anti-metabolites, intercalating antibiotics, growth factor inhibitors, cell cycle inhibitors, enzyme inhibitors, topoisomerase inhibitors, biological response modifiers, anti-hormones, angiogenesis inhibitors, and anti-androgens.

In some aspects the invention provides a method of treating diseases related to vasculogenesis or angiogenesis in a mammal that comprises administering to said mammal a therapeutically effective amount of a compound of the present invention, or a pharmaceutically acceptable salt, ester, prodrug, solvate, hydrate or derivative thereof. In some embodiments, said method is for treating a disease selected from the group consisting of tumor angiogenesis, chronic inflammatory disease such as rheumatoid arthritis, atherosclerosis, inflammatory bowel disease, skin diseases such as psoriasis, eczema, and scleroderma, diabetes, diabetic retinopathy, retinopathy of prematurity, age-related macular degeneration, hemangioma, glioma, melanoma, Kaposi's sarcoma and ovarian, breast, lung, pancreatic, prostate, colon and epidermoid cancer.

In some aspects patients that can be treated with compounds of the present invention, or pharmaceutically acceptable salt, ester, prodrug, solvate, hydrate or derivative of said compounds, according to the methods of this invention include, for example, patients that have been diagnosed as having psoriasis; restenosis; atherosclerosis; BPH; breast cancer such as a ductal carcinoma in duct tissue in a mammary gland, medullary carcinomas, colloid carcinomas, tubular carcinomas, and inflammatory breast cancer; ovarian cancer, including epithelial ovarian tumors such as adenocarcinoma in the ovary and an adenocarcinoma that has migrated from the ovary into the abdominal cavity; uterine cancer; cervical cancer such as adenocarcinoma in the cervix epithelial including squamous cell carcinoma and adenocarcinomas; prostate cancer, such as a prostate cancer selected from the following: an adenocarcinoma or an adenocarinoma that has migrated to the bone; pancreatic cancer such as epitheliod carcinoma in the pancreatic duct tissue and an adenocarcinoma in a pancreatic duct; bladder cancer such as a transitional cell carcinoma in urinary bladder, urothelial carcinomas (transitional cell carcinomas), tumors in the urothelial cells that line the bladder, squamous cell carcinomas, adenocarcinomas, and small cell cancers; leukemia such as acute myeloid leukemia (AML), acute lymphocytic leukemia, chronic lymphocytic leukemia, chronic myeloid leukemia, hairy cell leukemia, myelodysplasia, myeloproliferative disorders, acute myelogenous leukemia (AML), chronic myelogenous leukemia (CML), mastocytosis, chronic lymphocytic leukemia (CLL), multiple myeloma (MM), and myelodysplastic syndrome (MDS); bone cancer; lung cancer such as non-small cell lung cancer (NSCLC), which is divided into squamous cell carcinomas, adenocarcinomas, and large cell undifferentiated carcinomas, and small cell lung cancer; skin cancer such as basal cell carcinoma, melanoma, squamous cell carcinoma and actinic keratosis, which is a skin condition that sometimes develops into squamous cell carcinoma; eye retinoblastoma; cutaneous or intraocular (eye) melanoma; primary liver cancer (cancer that begins in the liver); kidney cancer; thyroid cancer such as papillary, follicular, medullary and anaplastic; AIDS-related lymphoma such as diffuse large B-cell lymphoma, B-cell immunoblastic lymphoma and small non-cleaved cell lymphoma; Kaposi's Sarcoma; viral-induced cancers including hepatitis B virus (HBV), hepatitis C virus (HCV), and hepatocellular carcinoma; human lymphotropic virus-type 1 (HTLV-I) and adult T-cell leukemia/lymphoma; and human papilloma virus (HPV) and cervical cancer; central nervous system cancers (CNS) such as primary brain tumor, which includes gliomas (astrocytoma, anaplastic astrocytoma, or glioblastoma multiforme), Oligodendroglioma, Ependymoma, Meningioma, Lymphoma, Schwannoma, and Medulloblastoma; peripheral nervous system (PNS) cancers such as acoustic neuromas and malignant peripheral nerve sheath tumor (MPNST) including neurofibromas and schwannomas, malignant fibrous cytoma, malignant fibrous histiocytoma, malignant meningioma, malignant mesothelioma, and malignant mixed Mullerian tumor; oral cavity and oropharyngeal cancer such as, hypopharyngeal cancer, laryngeal cancer, nasopharyngeal cancer, and oropharyngeal cancer; stomach cancer such as lymphomas, gastric stromal tumors, and carcinoid tumors; testicular cancer such as germ cell tumors (GCTs), which include seminomas and nonseminomas, and gonadal stromal tumors, which include Leydig cell tumors and Sertoli cell tumors; thymus cancer such as to thymomas, thymic carcinomas, Hodgkin disease, non-Hodgkin lymphomas carcinoids or carcinoid tumors; rectal cancer; and colon cancer.

In some aspects the invention provides a method of treating diabetes in a mammal that comprises administering to said mammal a therapeutically effective amount of a compound of the present invention, or a pharmaceutically acceptable salt, ester, prodrug, solvate, hydrate or derivative thereof.

In some aspects the invention provides a method of treating an inflammation disorder, including autoimmune diseases, in a mammal that comprises administering to said mammal a therapeutically effective amount of a compound of the present invention, or a pharmaceutically acceptable salt, ester, prodrug, solvate, hydrate or derivative thereof. Examples of autoimmune diseases includes but is not limited to acute disseminated encephalomyelitis (ADEM), Addison's disease, antiphospholipid antibody syndrome (APS), aplastic anemia, autoimmune hepatitis, coeliac disease, Crohn's disease, Diabetes mellitus (type 1), Goodpasture's syndrome, Graves' disease, Guillain-Barr syndrome (GBS), Hashimoto's disease, lupus erythematosus, multiple sclerosis, myasthenia gravis, opsoclonus myoclonus syndrome (OMS), optic neuritis, Ord's thyroiditis, oemphigus, polyarthritis, primary biliary cirrhosis, psoriasis, rheumatoid arthritis, Reiter's syndrome, Takayasu's arteritis, temporal arteritis (also known as “giant cell arteritis”), warm autoimmune hemolytic anemia, Wegener's granulomatosis, alopecia universalis, Chagas' disease, chronic fatigue syndrome, dysautonomia, endometriosis, hydradenitis suppurativa, interstitial cystitis, neuromyotonia, sarcoidosis, scleroderma, ulcerative colitis, vitiligo, and vulvodynia. Other disorders include bone-resorption disorders and thromobsis.

For instance, in some aspects the compounds described herein are used to treat encephalomyelitis. In other embodiments the compounds described herein are used for the treatment of obstructive pulmonary disease. Chronic obstructive pulmonary disease (COPD) is an umbrella term for a group of respiratory tract diseases that are characterized by airflow obstruction or limitation. Conditions included in this umbrella term are: chronic bronchitis, emphysema, and bronchiectasis.

In some aspects, the compounds described herein are used for the treatment of asthma. Also, the compounds described herein may be used for the treatment of endotoxemia and sepsis. In one embodiment, the compounds described herein are used to for the treatment of rheumatoid arthritis (RA). In yet another embodiment, the compounds described herein is used for the treatment of contact or atopic dermatitis. Contact dermatitis includes irritant dermatitis, phototoxic dermatitis, allergic dermatitis, photoallergic dermatitis, contact urticaria, systemic contact-type dermatitis and the like. Irritant dermatitis can occur when too much of a substance is used on the skin of when the skin is sensitive to certain substance. Atopic dermatitis, sometimes called eczema, is a kind of dermatitis, an atopic skin disease.

In another embodiment, compounds described herein may be used to treat acne.

In another embodiment, the compounds described herein may be used for the treatment of arteriosclerosis, including atherosclerosis. Arteriosclerosis is a general term describing any hardening of medium or large arteries. Arterosclerosis is a hardening of an artery specifically due to an atheromatous plaque.

In another embodiment, the compounds described herein may be used for the treatment of neurological disorders that accompany abnormal protein accumulation, including Alzheimer's disease.

In another embodiment, the compounds described herein may be used for the treatment of glomerulonephritis. Glomerulonephritis is a primary or secondary autoimmune renal disease characterized by inflammation of the glomeruli. It may be asymptomatic, or present with hematuria and/or proteinuria. There are many recognized types, divided in acute, subacute or chronic glomerulonephritis. Causes are infectious (bacterial, viral or parasitic pathogens), autoimmune or paraneoplastic.

In other embodiments, the compounds described herein may be used for the treatment of bursitis, lupus, acute disseminated encephalomyelitis (ADEM), Addison's disease, antiphospholipid antibody syndrome (APS), aplastic anemia, autoimmune hepatitis, coeliac disease, Crohn's disease, diabetes mellitus (type 1), goodpasture's syndrome, graves' disease, guillain-barre syndrome (GBS), hashimoto's disease, inflammatory bowel disease, lupus erythematosus, myasthenia gravis, opsoclonus myoclonus syndrome (OMS), optic neuritis, ord's thyroiditis, ostheoarthritis, uveoretinitis, pemphigus, polyarthritis, primary biliary cirrhosis, reiter's syndrome, takayasu's arteritis, temporal arteritis, warm autoimmune hemolytic anemia, Wegener's granulomatosis, alopecia universalis, chagas' disease, chronic fatigue syndrome, dysautonomia, endometriosis, hidradenitis suppurativa, interstitial cystitis, neuromyotonia, sarcoidosis, scleroderma, ulcerative colitis, vitiligo, vulvodynia, appendicitis, arteritis, arthritis, blepharitis, bronchiolitis, bronchitis, cervicitis, cholangitis, cholecystitis, chorioamnionitis, colitis, conjunctivitis, cystitis, dacryoadenitis, dermatomyositis, endocarditis, endometritis, enteritis, enterocolitis, epicondylitis, epididymitis, fasciitis, fibrositis, gastritis, gastroenteritis, gingivitis, hepatitis, hidradenitis, ileitis, iritis, laryngitis, mastitis, meningitis, myelitis, myocarditis, myositis, nephritis, omphalitis, oophoritis, orchitis, osteitis, otitis, pancreatitis, parotitis, pericarditis, peritonitis, pharyngitis, pleuritis, phlebitis, pneumonitis, proctitis, prostatitis, pyelonephritis, rhinitis, salpingitis, sinusitis, stomatitis, synovitis, tendonitis, tonsillitis, uveitis, vaginitis, vasculitis, or vulvitis.

The invention also relates to a method of treating a cardiovascular disease in a mammal that comprises administering to said mammal a therapeutically effective amount of a compound of the present invention, or a pharmaceutically acceptable salt, ester, prodrug, solvate, hydrate or derivative thereof. Examples of cardiovascular conditions include, but are not limited to, atherosclerosis, restenosis, vascular occlusion and carotid obstructive disease.

In another aspect, the present invention provides methods of disrupting the function of a ribosome through inhibition of elongation factors. The method includes contacting the ribosome with a function-disrupting amount of a compound of the invention. The invention further provides methods of modulating ribosome activity by contacting a ribosome with an amount of a compound of the invention sufficient to modulate the activity of the ribosome. “To modulate” can mean to inhibit or to activate ribosome activity. In some embodiments, the invention provides methods of inhibiting ribosome activity by contacting a ribosome with an amount of a compound of the invention sufficient to inhibit the activity of the ribosome. In some embodiments, the invention provides methods of inhibiting ribosome activity in a solution by contacting said solution with an amount of a compound of the invention sufficient to inhibit the activity of the ribosome in said solution. In some embodiments, the invention provides methods of inhibiting ribosome activity in a cell by contacting said cell with an amount of a compound of the invention sufficient to inhibit the activity of the ribosome in said cell. In some embodiments, the invention provides methods of inhibiting ribosome activity in a tissue by contacting said tissue with an amount of a compound of the invention sufficient to inhibit the activity of the ribosome in said tissue. In some embodiments, the invention provides methods of inhibiting ribosome activity in an organism by contacting said organism with an amount of a compound of the invention sufficient to inhibit the activity of the ribosome in said organism. In some embodiments, the invention provides methods of inhibiting ribosome activity in an animal by contacting said animal with an amount of a compound of the invention sufficient to inhibit the activity of the ribosome in said animal. In some embodiments, the invention provides methods of inhibiting ribosome activity in a mammal by contacting said mammal with an amount of a compound of the invention sufficient to inhibit the activity of the ribosome in said mammal. In some embodiments, the invention provides methods of inhibiting ribosome activity in a human by contacting said human with an amount of a compound of the invention sufficient to inhibit the activity of the ribosome in said human. In some embodiments, the % of ribosome activity after contacting a ribosome with a compound of the invention is less than 10, 20, 30, 40, 50, 60, 70, 80 or 90% of the ribosome activity in the absence of said contacting step.

The present invention provides methods of treating a disease mediated by ribosome activity (e.g. Elongation Factor 1(EF1) or Elongation Factor 2 (EF2)) in a subject in need of such treatment. The method includes administering to the subject a therapeutically effective amount of a compound of the invention.

The present chemical entities, pharmaceutical compositions and methods provide manners of modulating the catalytic activity of a ribosome. The method includes the step of contacting the ribosome with an activity modulating amount an affinity pocket binding chemical entity antagonist. Also provided are methods of treating a condition or disorder mediated by ribosome activity in a subject in need of such treatment. The method includes administering to the subject a therapeutically effective amount of a chemical entity antagonist.

### Combination Treatment

In some aspects the present invention also provides methods for combination therapies in which an agent known to modulate other pathways, or other components of the same pathway, or even overlapping sets of target enzymes are used in combination with a compound of the present invention, or a pharmaceutically acceptable salt, ester, prodrug, solvate, hydrate or derivative thereof. In one aspect, such therapy includes but is not limited to the combination of compounds of this invention with chemotherapeutic agents, therapeutic antibodies, and radiation treatment, to provide a synergistic therapeutic effect.

Specifically, in one aspect, this invention also relates to a pharmaceutical composition for inhibiting abnormal cell growth or protein accumulation in a mammal which comprises an amount of a compound of the present invention, or a pharmaceutically acceptable salt, ester, prodrug, solvate, hydrate or derivative thereof, in combination with an amount of an anti-cancer agent (e.g. a chemotherapeutic agent), wherein the amounts of the compound, salt, ester, prodrug, solvate, hydrate or derivative, and of the chemotherapeutic are together effective in inhibiting abnormal cell growth. Many chemotherapeutics are presently known in the art and can be used in combination with the compounds of the invention.

In some embodiments, the chemotherapeutic is selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, angiogenesis inhibitors, and anti-androgens.

A wide variety of anti-cancer agents can be employed in combination. Non-limiting examples are chemotherapeutic agents, cytotoxic agents, and non-peptide small molecules such as Gleevec (imatinib mesylate), Velcade (bortezomib), Casodex (bicalutamide), Iressa (gefitinib), and Adriamycin as well as ahost of chemotherapeutic agents. Non-limiting examples of chemotherapeutic agents include alkylating agents such as thiotepa and cyclosphosphamide (CYTOXAN™); alkyl sulfonates such as busulfan, improsulfan and piposulfan; aziridlnes such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinopbilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfomithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK.R™; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel (TAXOL™, Bristol-Myers Squibb Oncology, Princeton, N.J.) and docetaxel (TAXOTERE™, Rhone-Poulenc Rorer, Antony, France); retinoic acid; esperamicins; capecitabine; and pharmaceutically acceptable salts, acids or derivatives of any of the above. Also included as suitable chemotherapeutic cell conditioners are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY 117018, onapristone, and toremifene (Fareston); and anti-androgens such as flutamide, nilutamide, bicalutamide, leuprolide, and goserelin; chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; camptothecin-11 (CPT-11); topoisomerase inhibitor RFS 2000; difluoromethylornithine (DMFO). This invention further relates to a method for inhibiting abnormal cell growth in a mammal or treating a hyperproliferative disorder which method comprises administering to the mammal an amount of a compound of the present invention, or a pharmaceutically acceptable salt, ester, prodrug, solvate, hydrate or derivative thereof, in combination with radiation therapy, wherein the amounts of the compound, salt, ester, prodrug, solvate, hydrate or derivative, is in combination with the radiation therapy effective in inhibiting abnormal cell growth or treating the hyperproliferative disorder in the mammal Techniques for administering radiation therapy are known in the art, and these techniques can be used in the combination therapy described herein. The administration of one or more of the compounds of the invention in this combination therapy can be determined as described herein.

In some embodiments bouvardin, streptovitacin A, or a bouvardin derivative is used in combination with taxanes, e.g. paclitaxel (TAXOL™, Bristol-Myers Squibb Oncology, Princeton, N.J.) and docetaxel (TAXOTERE™, Rhone-Poulenc Rorer, Antony, France). In some embodiments bouvardin or a bouvardin derivative is used in combination with paclitaxel. In some embodiments a composition comprising paclitaxel and bouvardin or a bouvardin derivative is administered. In some embodiments bouvardin or a bouvardin derivative is used in combination with DHA-paclitaxel. In some embodiments a composition comprising DHA-paclitaxel and bouvardin, streptovitacin A, or a bouvardin derivative is administered. In some embodiments bouvardin or a bouvardin derivative is used in combination with tumor-activated Taxol prodrugs or paclitaxel bonded to a polyglutamate polymer.

In some embodiments, use of compounds of the present invention may allow for the reduction of the dosage of Taxol in the treatment of cancers. In some embodiments, the amount of Taxol administered intravenously to treat breast carcinoma, when combined with one or more compounds of the present invention, is about 175 mg/m2 over 3 hours, or about 165 mg/m2 over 3 hours, or about 155 mg/m2 over 3 hours, or about 145 mg/m2 over 3 hours, or about 135 mg/m2 over 3 hours, or about 125 mg/m2 over 3 hours, or about 115 mg/m2 over 3 hours, or about 105 mg/m2 over 3 hours, or about 95 mg/m2 over 3 hours, or about 85 mg/m2 over 3 hours, or about 75 mg/m2 over 3 hours, or about 65 mg/m2 over 3 hours, or about 55 mg/m2 over 3 hours, or about 45 mg/m2 over 3 hours, or about 35 mg/m2 over 3 hours, or about 25 mg/m2 over 3 hours, or about 15 mg/m2 over 3 hours, or about 5 mg/m2 over 3 hours, or about 1 mg/m2 over 3 hours.

In some embodiments, the amount of Taxol administered intravenously to treat non-small cell lung carcinoma, when combined with one or more compounds of the present invention, is about 135 mg/m2 over 24 hours, or about 125 mg/m2 over 3 hours, or about 115 mg/m2 over 24 hours, or about 105 mg/m2 over 24 hours, or about 95 mg/m2 over 3 hours, or about 85 mg/m2 over 24 hours, or about 75 mg/m2 over 24 hours, or about 65 mg/m2 over 24 hours, or about 55 mg/m2 over 24 hours, or about 45 mg/m2 over 24 hours, or about 35 mg/m2 over 24 hours, or about 25 mg/m2 over 24 hours, or about 15 mg/m2 over 24 hours, or about 5 mg/m2 over 24 hours, or about 1 mg/m2 over 24 hours.

In further embodiments, treatment with Taxol and one or more compounds of this invention can be followed by intravenous administration of cis-platin. In some embodiments, the amount of cis-platin administered is about 75 mg/m2, or about 65 mg/m2, or about 55 mg/m2, or about 45 mg/m2, or about 35 mg/m2, or about 25 mg/m2, or about 15 mg/m2, or about 5 mg/m2, or about 1 mg/m2.

In some embodiments other taxanes are administered as described for Taxol.

In some aspects the anti-cancer agents and one or more of the compounds of the invention are administered simultaneously. In some embodiments the anti-cancer agents and one or more of the compounds of the invention are administered in the same formulation. In some embodiments the anti-cancer agents and one or more of the compounds of the invention are administered in a staggered fashion, for instance every other day or at different meal times. In some embodiments the anti-cancer agents and one or more of the compounds of the invention are administered as two separate formulations administered at different times. In some embodiments the anti-cancer agents and one or more of the compounds of the invention are administered as two separate formulations administered at similar times.

In some aspects the invention provides for method for instructing a patient or medical professional regarding the proper dosage of a compound of the invention to be used in combination with one or more additional anti-cancer agents. In some embodiments the invention provides for method for instructing a patient regarding the proper compound of the invention to be used in combination with a particular anti-cancer agent.

In various aspects radiation therapy is administered through one of several methods, or a combination of methods, including without limitation external-beam therapy, internal radiation therapy, implant radiation, stereotactic radiosurgery, systemic radiation therapy, radiotherapy and permanent or temporary interstitial brachytherapy. The term “brachytherapy,” as used herein, refers to radiation therapy delivered by a spatially confined radioactive material inserted into the body at or near a tumor or other proliferative tissue disease site. The term is intended without limitation to include exposure to radioactive isotopes (e.g. At-211, 1-131, 1-125, Y-90, Re-186, Re-188, Sm-153, Bi-212, P-32, and radioactive isotopes of Lu), Suitable radiation sources for use as a cell conditioner of the present invention include both solids and liquids. By way of non-limiting example, the radiation source can be a radionuclide, such as 1-125, 1-131, Yb-169, Ir-192 as a solid source, 1-125 as a solid source, or other radionuclides that emit photons, beta particles, gamma radiation, or other therapeutic rays. The radioactive material can also be a fluid made from any solution of radionuclide(s), e.g., a solution of I-125 or I-131, or a radioactive fluid can be produced using a slurry of a suitable fluid containing small particles of solid radionuclides, such as Au-198, Y-90. Moreover, the radionuclide(s) can be embodied in a gel or radioactive micro spheres.

In some embodiments bouvardin, streptovitacin A, or bouvardin derivative synergizes with radiation and enhances the effect of radiation in a human cancer. Without being bound by theory it is thought that inhibitors of translation elongation such as bouvardin are a new class of molecules with potential to improve the outcome of radiation therapy in clinical settings. Accordingly compounds of the invention are, in various embodiments administered to a patient before, during, or after radiation therapy.

Without being limited by any theory, the compounds of the present invention render abnormal cells more sensitive to treatment with radiation for purposes of killing and/or inhibiting the growth of such cells.

Without being limited by any theory, the compounds of the present invention render abnormal cells more sensitive to treatment with a chemotherapy agent for purposes of killing and/or inhibiting the growth of such cells.

Accordingly, aspects of this invention further relates to a method for sensitizing abnormal cells in a mammal to treatment with radiation which comprises administering to the mammal an amount of a compound of the present invention or pharmaceutically acceptable salt, ester, prodrug, solvate, hydrate or derivative thereof, which amount is effective is sensitizing abnormal cells to treatment with radiation. The amount of the compound, salt, or solvate in this method can be determined according to the means for ascertaining effective amounts of such compounds described herein.

The invention also relates to a method of and to a pharmaceutical composition of inhibiting abnormal cell growth in a mammal which comprises an amount of a compound of the present invention, or a pharmaceutically acceptable salt, ester, prodrug, solvate, hydrate or derivative thereof, or an isotopically-labeled derivative thereof, and an amount of one or more substances selected from anti-angiogenesis agents, signal transduction inhibitors, and antiproliferative agents.

Anti-angiogenesis agents, such as MMP-2 (matrix-metalloprotienase 2) inhibitors, MMP-9 (matrix-metalloprotienase 9) inhibitors, and COX-11 (cyclooxygenase 11) inhibitors, can be used in conjunction with a compound of the present invention and pharmaceutical compositions described herein. Examples of useful COX-II inhibitors include CELEBREX™ (alecoxib), valdecoxib, and rofecoxib. Examples of useful matrix metalloproteinase inhibitors are described in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606,046 (published Jul. 13, 1994), European Patent Publication 931,788 (published Jul. 28, 1999), WO 90/05719 (published May 31, 1990), WO 99/52910 (published Oct. 21, 1999), WO 99/52889 (published Oct. 21, 1999), WO 99/29667 (published Jun. 17, 1999), PCT International Application No. PCT/IB98/01113 (filed Jul. 21, 1998), European Patent Application No. 99302232.1 (filed Mar. 25, 1999), Great Britain Patent Application No. 9912961.1 (filed Jun. 3, 1999), U.S. Provisional Application No. 60/148,464 (filed Aug. 12, 1999), U.S. Pat. No. 5,863,949 (issued Jan. 26, 1999), U.S. Pat. No. 5,861,510 (issued Jan. 19, 1999), and European Patent Publication 780,386 (published Jun. 25, 1997), all of which are incorporated herein in their entireties by reference. Preferred MMP-2 and MMP-9 inhibitors are those that have little or no activity inhibiting MMP-I. Alsoagents are those that selectively inhibit MMP-2 and/or AMP-9 relative to the other matrix-metalloproteinases (i. e., MAP-I, MMP-3, MMP-4, MMP-5, MMP-6, MMP-7, MMP-8, MMP-10, MMP-I1, MMP-12, and MMP-13). Some specific examples of MMP inhibitors useful in the present invention are AG-3340, RO 32-3555, and RS 13-0830.

In some embodiments the invention also relates to a method of and to a pharmaceutical composition of treating a cardiovascular disease in a mammal which comprises an amount of a compound of the present invention, or a pharmaceutically acceptable salt, ester, prodrug, solvate, hydrate or derivative thereof, or an isotopically-labeled derivative thereof, and an amount of one or more therapeutic agents use for the treatment of cardiovascular diseases.

Examples for use in cardiovascular disease applications are anti-thrombotic agents, e.g., prostacyclin and salicylates, thrombolytic agents, e.g., streptokinase, urokinase, tissue plasminogen activator (TPA) and anisoylated plasminogen-streptokinase activator complex (APSAC), anti-platelets agents, e.g., acetyl-salicylic acid (ASA) and clopidrogel, vasodilating agents, e.g., nitrates, calcium channel blocking drugs, antiproliferative agents, e.g., colchicine and alkylating agents, intercalating agents, growth modulating factors such as interleukins, transformation growth factor-beta and congeners of platelet derived growth factor, monoclonal antibodies directed against growth factors, anti-inflammatory agents, both steroidal and non-steroidal, and other agents that can modulate vessel tone, function, arteriosclerosis, and the healing response to vessel or organ injury post intervention. Antibiotics can also be included in combinations or coatings comprised by the invention. Moreover, a coating can be used to effect therapeutic delivery focally within the vessel wall. By incorporation of the active agent in a swellable polymer, the active agent will be released upon swelling of the polymer.

The compounds of the invention may be formulated or administered in conjunction with other agents that act to relieve the symptoms of inflammatory conditions such as encephalomyelitis, asthma, and the other diseases described herein. These agents include non-steroidal anti-inflammatory drugs (NSAIDs), e.g. acetylsalicylic acid; ibuprofen; naproxen; indomethacin; nabumetone; tolmetin; etc. Corticosteroids are used to reduce inflammation and suppress activity of the immune system. The most commonly prescribed drug of this type is Prednisone. Chloroquine (Aralen) or hydroxychloroquine (Plaquenil) may also be very useful in some individuals with lupus. They are most often prescribed for skin and joint symptoms of lupus. Azathioprine (Imuran) and cyclophosphamide (Cytoxan) suppress inflammation and tend to suppress the immune system. Other agents, e.g. methotrexate and cyclosporin are used to control the symptoms of lupus. Anticoagulants are employed to prevent blood from clotting rapidly. They range from aspirin at very low dose which prevents platelets from sticking, to heparin/coumadin.

The compounds describe herein may be formulated or administered in conjunction with liquid or solid tissue barriers also known as lubricants. Examples of tissue barriers include, but are not limited to, polysaccharides, polyglycans, seprafilm, interceed and hyaluronic acid.

In some aspects medicaments are administered in conjunction with the compounds described herein. Such medicaments include any suitable drugs usefully delivered by inhalation for example, analgesics, e.g. codeine, dihydromorphine, ergotamine, fentanyl or morphine; anginal preparations, e.g. diltiazem; antiallergics, e.g. cromoglycate, ketotifen or nedocromil; anti-infectives, e.g. cephalosporins, penicillins, streptomycin, sulphonamides, tetracyclines or pentamidine; antihistamines, e.g. methapyrilene; antiinflammatories, e.g. beclomethasone, flunisolide, budesonide, tipredane, triamcinolone acetonide or fluticasone; antitussives, e.g. noscapine; bronchodilators, e.g. ephedrine, adrenaline, fenoterol, formoterol, isoprenaline, metaproterenol, phenylephrine, phenylpropanolamine, pirbuterol, reproterol, rimiterol, salbutamol, salmeterol, terbutalin, isoetharine, tulobuterol, orciprenaline or (−)-4-amino-3,5-dichloro-α-[[[6-[2-(2-pyridinyl)ethoxy]hexyl]-amino]methyl]benzenemethanol; diuretics, e.g. amiloride; anticholinergics e.g. ipratropium, atropine or oxitropium; hormones, e.g. cortisone, hydrocortisone or prednisolone; xanthines e.g. aminophylline, choline theophyllinate, lysine theophyllinate or theophylline; and therapeutic proteins and peptides, e.g. insulin or glucagon. It will be clear to a person skilled in the art that, where appropriate, the medicaments maybe used in the form of salts (e.g. as alkali metal or amine salts or as acid addition salts) or as esters (e.g. lower alkyl esters) or as solvates (e.g. hydrates) to optimize the activity and/or stability of the medicament.

Other exemplary therapeutic agents useful for a combination therapy include but are not limited to agents as described above, radiation therapy, hormone antagonists, hormones and their releasing factors, thyroid and antithyroid drugs, estrogens and progestins, androgens, adrenocorticotropic hormone; adrenocortical steroids and their synthetic analogs; inhibitors of the synthesis and actions of adrenocortical hormones, insulin, oral hypoglycemic agents, and the pharmacology of the endocrine pancreas, agents affecting calcification and bone turnover: calcium, phosphate, parathyroid hormone, vitamin D, calcitonin, vitamins such as water-soluble vitamins, vitamin B complex, ascorbic acid, fat-soluble vitamins, vitamins A, K, and E, growth factors, cytokines, chemokines, muscarinic receptor agonists and antagonists; anticholinesterase agents; agents acting at the neuromuscular junction and/or autonomic ganglia; catecholamines, sympathomimetic drugs, and adrenergic receptor agonists or antagonists; and 5-hydroxytryptamine (5-HT, serotonin) receptor agonists and antagonists.

Therapeutic agents can also include agents for pain and inflammation such as histamine and histamine antagonists, bradykinin and bradykinin antagonists, 5-hydroxytryptamine (serotonin), lipid substances that are generated by biotransformation of the products of the selective hydrolysis of membrane phospholipids, eicosanoids, prostaglandins, thromboxanes, leukotrienes, aspirin, nonsteroidal anti-inflammatory agents, analgesic-antipyretic agents, agents that inhibit the synthesis of prostaglandins and thromboxanes, selective inhibitors of the inducible cyclooxygenase, selective inhibitors of the inducible cyclooxygenase-2, autacoids, paracrine hormones, somatostatin, gastrin, cytokines that mediate interactions involved in humoral and cellular immune responses, lipid-derived autacoids, eicosanoids, β-adrenergic agonists, ipratropium, glucocorticoids, methylxanthines, sodium channel blockers, opioid receptor agonists, calcium channel blockers, membrane stabilizers and leukotriene inhibitors.

Additional therapeutic agents contemplated herein include diuretics, vasopressin, agents affecting the renal conservation of water, rennin, angiotensin, agents useful in the treatment of myocardial ischemia, anti-hypertensive agents, angiotensin converting enzyme inhibitors, β-adrenergic receptor antagonists, agents for the treatment of hypercholesterolemia, and agents for the treatment of dyslipidemia.

Other therapeutic agents contemplated include drugs used for control of gastric acidity, agents for the treatment of peptic ulcers, agents for the treatment of gastroesophageal reflux disease, prokinetic agents, antiemetics, agents used in irritable bowel syndrome, agents used for diarrhea, agents used for constipation, agents used for inflammatory bowel disease, agents used for biliary disease, agents used for pancreatic disease, herapeutic agents used to treat protozoan infections, drugs used to treat malaria, amebiasis, giardiasis, trichomoniasis, trypanosomiasis, and/or leishmaniasis, and/or drugs used in the chemotherapy of helminthiasis. Other therapeutic agents include antimicrobial agents, sulfonamides, trimethoprim-sulfamethoxazole quinolones, and agents for urinary tract infections, penicillins, cephalosporins, and other, β-Lactam antibiotics, an agent comprising an aminoglycoside, protein synthesis inhibitors, drugs used in the chemotherapy of tuberculosis, mycobacterium avium complex disease, and leprosy, antifungal agents, antiviral agents including nonretroviral agents and antiretroviral agents.

Examples of therapeutic antibodies that can be combined with compounds of this invention include but are not limited to anti-receptor tyrosine kinase antibodies (cetuximab, panitumumab, trastuzumab), anti CD20 antibodies (rituximab, tositumomab), and other antibodies such as alemtuzumab, bevacizumab, and gemtuzumab. Moreover, therapeutic agents used for immunomodulation, such as immunomodulators, immunosuppressive agents, tolerogens, and immunostimulants are contemplated by the methods herein. In addition, therapeutic agents acting on the blood and the blood-forming organs, hematopoietic agents, growth factors, minerals, and vitamins, anticoagulant, thrombolytic, and antiplatelet drugs.

Further therapeutic agents that can be combined with one or more compounds of this invention may be found in Goodman and Gilman's “The Pharmacological Basis of Therapeutics,” Tenth Edition, edited by Hardman, Limbird and Gilman or the “Physician's Desk Reference”, Thomson Reuters; 63rd edition, both of which are incorporated herein by reference in their entirety.

The compounds described herein can be used in combination with the agents disclosed herein or other suitable agents, depending on the condition being treated. Hence, in some embodiments the compounds of the invention will be co-administered with other agents as described above. When used in combination therapy, the compounds described herein may be administered with the second agent simultaneously or separately. This administration in combination can include simultaneous administration of the two agents in the same dosage form, simultaneous administration in separate dosage forms, and separate administration. That is, a compound described herein and any of the agents described above can be formulated together in the same dosage form and administered simultaneously. Alternatively, a compound of the present invention and any of the agents described above can be simultaneously administered, wherein both the agents are present in separate formulations. In another alternative, a compound of the present invention can be administered just followed by and any of the agents described above, or vice versa. In the separate administration protocol, a compound of the present invention and any of the agents described above may be administered a few minutes apart, or a few hours apart, or a few days apart.

### Administration

In some aspects of the invention the administration of the compounds of the present invention is effected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g. transdermal application), rectal administration, via local delivery by catheter or stent. Compounds can also be administered intraadiposally or intrathecally.

In some aspects of the invention the amount of the compound administered is dependent on the mammal being treated, the severity of the disorder or condition, the rate of administration, the disposition of the compound and the discretion of the prescribing physician.

In some aspects of the invention, the compounds are applied as a sole therapy or may involve one or more other anti-tumor substances, for example those selected from, mitotic inhibitors, for example vinblastine or a taxane; alkylating agents, for example cis-platin, carboplatin and cyclophosphamide; anti-metabolites, for example 5-fluorouracil, cytosine arabinside and hydroxyurea; growth factor inhibitors; cell cycle inhibitors; intercalating antibiotics, for example adriamycin and bleomycin; enzymes, for example, interferon; and anti-hormones, for example anti-estrogens such as Nolvadex™ (tamoxifen) or, for example anti-androgens such as Casodex™ (4′-cyano-3-(4-fluorophenylsulphonyl)-2-hydroxy-2-methyl-3′-(trifluoromethyl) propionanilide). Such conjoint treatment may be achieved by way of the simultaneous, sequential or separate dosing of the individual components of treatment.

In some embodiments, a compound of the invention is administered in a single dose. Typically, such administration will be by injection, e.g., intravenous injection, in order to introduce the agent quickly. However, other routes may be used as appropriate. A single dose of a compound of the invention may also be used for treatment of an acute condition.

In some embodiments, a compound of the invention is administered in multiple doses. Dosing may be about once, twice, three times, four times, five times, six times, or more than six times per day. Dosing may be about once a month, once every two weeks, once a week, or once every other day. In another embodiment a compound of the invention and another agent are administered together about once per day to about 6 times per day. In another embodiment the administration of a compound of the invention and an agent continues for less than about 7 days. In yet another embodiment the administration continues for more than about 6, 10, 14, 28 days, two months, six months, or one year. In some cases, continuous dosing is achieved and maintained as long as necessary.

Administration of the agents of the invention may continue as long as necessary. In some embodiments, an agent of the invention is administered for more than 1, 2, 3, 4, 5, 6, 7, 14, or 28 days. In some embodiments, an agent of the invention is administered for less than 28, 14, 7, 6, 5, 4, 3, 2, or 1 day. In some embodiments, an agent of the invention is administered chronically on an ongoing basis, e.g., for the treatment of chronic effects.

An effective amount of a compound of the invention may be administered in either single or multiple doses by any of the accepted modes of administration of agents having similar utilities, including rectal, buccal, intranasal and transdermal routes, by intra-arterial injection, intravenously, intraperitoneally, parenterally, intramuscularly, subcutaneously, orally, topically, or as an inhalant.

The compositions of the invention may also be delivered via an impregnated or coated device such as a stent, for example, or an artery-inserted cylindrical polymer. Such a method of administration may, for example, aid in the prevention or amelioration of restenosis following procedures such as balloon angioplasty. Without being bound by theory, compounds of the invention may slow or inhibit the migration and proliferation of smooth muscle cells in the arterial wall which contribute to restenosis. A compound of the invention may be administered, for example, by local delivery from the struts of a stent, from a stent graft, from grafts, or from the cover or sheath of a stent. In some embodiments, a compound of the invention is admixed with a matrix. Such a matrix may be a polymeric matrix, and may serve to bond the compound to the stent. Polymeric matrices suitable for such use, include, for example, lactone-based polyesters or copolyesters such as polylactide, polycaprolactonglycolide, polyorthoesters, polyanhydrides, polyaminoacids, polysaccharides, polyphosphazenes, poly (ether-ester) copolymers (e.g. PEO-PLLA); polydimethylsiloxane, poly(ethylene-vinylacetate), acrylate-based polymers or copolymers (e.g. polyhydroxyethyl methylmethacrylate, polyvinyl pyrrolidinone), fluorinated polymers such as polytetrafluoroethylene and cellulose esters. Suitable matrices may be nondegrading or may degrade with time, releasing the compound or compounds. Compounds of the invention may be applied to the surface of the stent by various methods such as dip/spin coating, spray coating, dip-coating, and/or brush-coating. The compounds may be applied in a solvent and the solvent may be allowed to evaporate, thus forming a layer of compound onto the stent. Alternatively, the compound may be located in the body of the stent or graft, for example in microchannels or micropores. When implanted, the compound diffuses out of the body of the stent to contact the arterial wall. Such stents may be prepared by dipping a stent manufactured to contain such micropores or microchannels into a solution of one or more of the compounds of the invention in a suitable solvent, followed by evaporation of the solvent. Excess drug on the surface of the stent may be removed via an additional brief solvent wash. In yet other embodiments, compounds of the invention may be covalently linked to a stent or graft. A covalent linker may be used which degrades in vivo, leading to the release of one or more of the compounds of the invention. Any bio-labile linkage may be used for such a purpose, such as ester, amide or anhydride linkages. Compounds of the invention may additionally be administered intravascularly from a balloon used during angioplasty. Extravascular administration of the compounds via the pericard or via advential application of formulations of the invention may also be performed to decrease restenosis. The compounds of the invention may be administered in dosages as described herein (see, e.g., Compositions). It is known in the art that due to intersubject variability in compound pharmacokinetics, individualization of dosing regimen is necessary for optimal therapy. Dosing for a compound of the invention may be found by routine experimentation.

When a compound of the invention, is administered in a composition that comprises one or more agents, and the agent has a shorter half-life than one or more of the compounds of the invention unit dose forms of the agent and one or more of the compounds of the invention may be adjusted accordingly. See e.g., “Pharmaceutical compositions for oral administration.” The subject pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical carrier or excipient and a compound according to the invention as an active ingredient. In addition, it may include other medicinal or pharmaceutical agents, carriers, adjuvants, etc. Exemplary parenteral administration forms include solutions or suspensions of active compound in sterile aqueous solutions, for example, aqueous propylene glycol or dextrose solutions. Such dosage forms can be suitably buffered, if desired.

The activity of the compounds of the present invention may be determined by the procedures described in the examples below.

### EXAMPLES

### Example 1

Streptovitacin A synergizes with radiation.

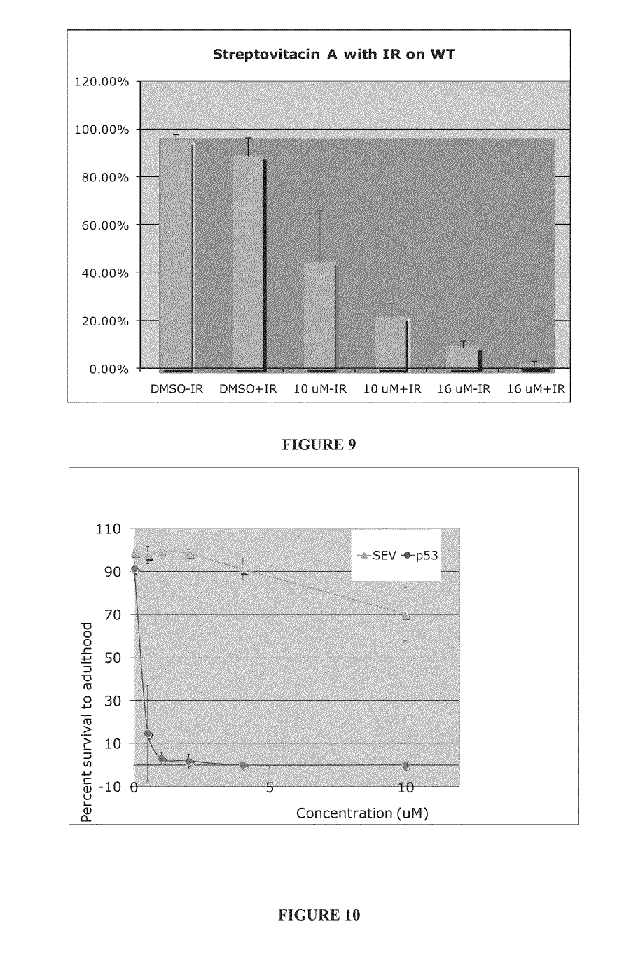

Testing with and without irradiation shows that streptovitacin A and ionizing radiation act synergistically to kill wild type Drosophila larvae. FIG. 9 depicts the combined effect. Amounts of Streptovitacin A are given in μM, as shown on the x-axis label. “μM-IR” means no irradiation was used. “μM+IR” means both irradiation and Streptovitacin A were used. Amount of larvae killed is given as a percentage.

### Example 2

Streptovitacin A is better at killing p53 mutant over wild type Drosophila larvae.

FIG. 10 depicts that at a wide range of doses the fraction of p53 mutant larvae that survive to adulthood is lower than the fraction of wild type larvae that survive to adulthood (the concentration given is that of Streptovitacin A).

### Example 3

Streptovitacin A synergizes with taxol

The effect of streptovitacin A on human cancer cells alone and in combination with taxol was tested.

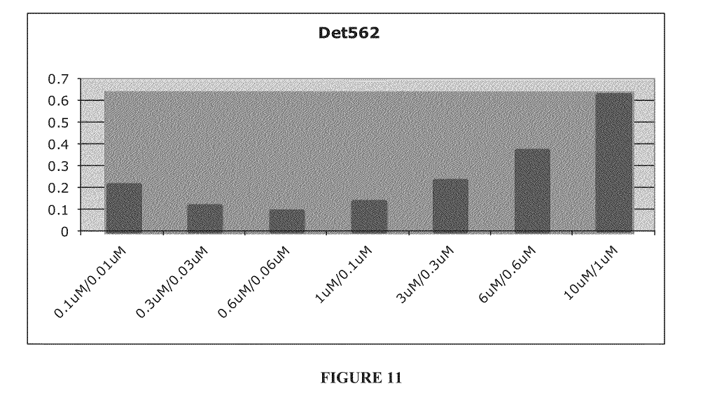

FIG. 11 depicts that at a wide range of doses the two drugs are synergistic (CI<1) in Det562 Head and Neck Squamous Cell Carcinoma cells.

### Example 4

Drosophila screen identifies 16 natural products that enhance the effect of radiation.

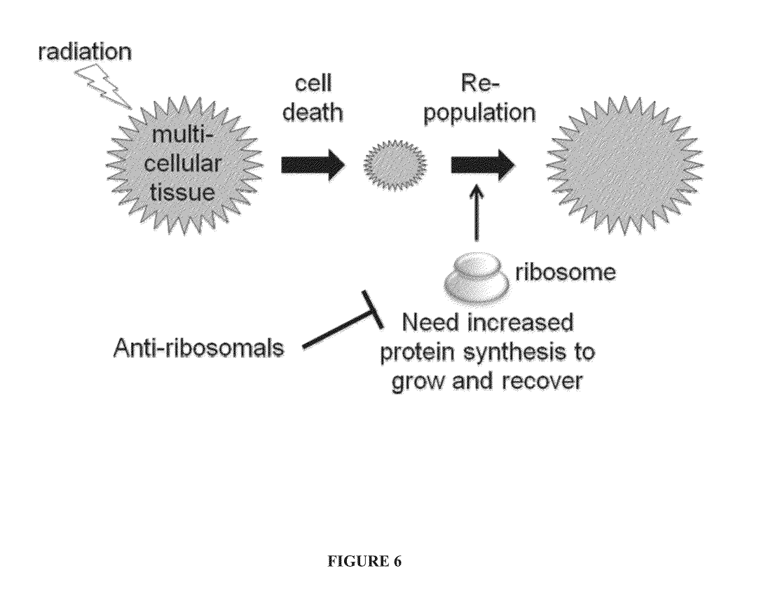

FIG. 6 depicts a rationale for why inhibition of protein synthesis enhances radiation therapy. Screens through two NCI libraries yielded anti-ribosomal agents. Bouvardin and streptovitacin A are two anti-ribosomals which inhibit translation elongation identified using the screen.

The Diversity Set I from the NCI-DTP, which is comprised of 1990 molecules chosen for chemical structural diversity and not for biological activity was screened using the Drosophila screen (U.S. Pat. No. 7,695,899). The screen yielded 4 molecules that enhanced the killing effects of radiation in Drosophila Chk1 homozygous null mutants. Diversity Set I includes 42 molecules of natural origin or 2% of the total, yet all four molecules identified in the Drosophila screen were of natural origin (Table 1).

Table 1. (below) depicts a summary of hits from two screens. A total of 2225 molecules in Diversity Set I (n=1990) and Natural Products Set (n=235) libraries from the NCI-DTP were screened for agents that increased the sensitivity of Drosophila Chk1 or p53 mutant larvae to radiation. Sixteen reproducible hits are shown along with their NSC number, common name, and most commonly attributed mechanism of action. MT=microtubule; DS=double strand; topo=topoisomerase; NT=nucleotide

TABLE 1

| NSC | Common Name | Mechanism/Structure |
|---|---|---|
| 757 | Colchicine (2) | MT Inhibitor |
| 292222 | Maytansine (3) | MT Inhibitor |
| 67574 | Vincristine (2) | MT Inhibitor |
| 259968 | Bouvardin (3) | Protein Synthesis Inhibitor |
| 325319 | Didemnin B (2) | Protein Synthesis Inhibitor |
| 39147 | Streptovitacin A (2) | Protein Synthesis Inhibitor |
| 106408 | Anthramycin (2) | DNA anti-metabolite: forms DNA adducts |
| 125006 | Bleomycin (2) | DNA anti-metabolite: causes DS breaks |
| 94600 | Camptothecin (2) | DNA anti-metabolite: topo I inhibitor |
| 639174 | 20-S | DNA anti-metabolite: topo I inhibitor |
| | Camptothecin (1) | |
| 609699 | Topotecan (1) | DNA anti-metabolite: topo I inhibitor |
| 200695 | Ftorafur (2) | DNA anti-metabolite: NT analog |
| 3053 | Actinomycin D (2) | Transcription inhibitor |
| 19941 | Guaiol (2) | Unknown |
| 28693 | Monocrotaline (2) | Toxic Alkaloid |
| 114572 | Ryanodine (2) | Alkaloid/Insecticide |

(1) Identified in the Diversity Set I screen
(2) Identified in the Natural Product Set screen
(3) Identified in both screens

The Natural Product Set, also from the NCI-DTP, which contains 235 small molecules derived from natural sources was selected next. These molecules were identified initially in extracts from plants, microbes and marine organisms, but are now synthesized to sufficient purity for distribution by the NCI as plated sets. Drosophila Chk1 and p53 homozygous null mutant larvae were used in the screen, in order to mimic the loss of p53 and checkpoint controls frequently found in human cancers. The screen through the Natural Product Set yielded 14 molecules, translating into a hit rate of 14/235 or 6%. This is higher than the hit rate from Diversity Set I (4 of 1990 or 0.2%), which includes compounds of both natural and synthetic origins. Without being bound by theory these results support the hypothesis that compounds of natural origin are particularly efficacious in the Drosophila screening model.

Sixteen hits from the two screens (two were found in both screens) fall into three major classes according to their mode of action (Table 1): microtubule depolymerizing agents, DNA anti-metabolites (Topoisomerase I inhibitors, nucleotide analogs, DNA damaging agents, etc), and protein synthesis inhibitors. Several microtubule poisons and DNA anti-metabolites are among FDA-approved anticancer agents. Microtubule poisons and DNA anti-metabolites are also among agents known to synergize with radiation in preclinical models of human cancer. For example, Topotecan and its analogs synergize with radiation in cells from human squamous cell carcinoma, melanoma, non-small cell lung carcinoma, malignant gliomas and small cell lung cancer. Synergy between Topotecan and radiation has been confirmed in xenografts of Rhabdomyosarcoma. Microtubule poisons and DNA anti-metabolites are also among agents used in combination therapy with radiation in treatment of human cancer. These lines of evidence support the idea that Drosophila can be used to find agents that are efficacious in human cancers.

The third class of hits from the Drosophila screen is comprised of three inhibitors of protein synthesis that are not structurally related. Didemnin B (NSC 325319) and analogs were originally extracted from tunicates or ‘sea squirts’, a marine invertebrate. Streptovitacin A (NSC 39147) and analogs were originally extracted from soil bacteria. Bouvardin (NSC 259968) and analogs were originally extracted from the plant Bouvardia ternifolia and related species. All three inhibit the elongation step of translation.

### Example 5

The efficacy of the protein synthesis inhibitors found in the Drosophila screen in pre-clinical mammalian cancer models was assessed.

Bouvardin blocks polypeptide chain elongation by inhibiting both the GTP and EF-2-dependent translocation of peptidyl-tRNA and the binding of aminoacyl-tRNA to the 80S ribosome. Bouvardin can cause arrest throughout the cell cycle marked it as a poor candidate for a useful chemotherapeutic agent (Tobey R A, Orlicky D J, Deaven L L, Rall L B, Kissane R J. Effects of Bouvardin (NSC 259968), a cyclic hexapeptide from Bouvardia ternifolia, on the progression capacity of cultured Chinese hamster. Cancer research 1978 December; 38(12):4415-21.). While Bouvardin may not be effective on its own, it still has the potential to synergize with other anti-cancer agents. Identification of Bouvardin in the Drosophila screen suggests that it may increase the effect of radiation in certain contexts.

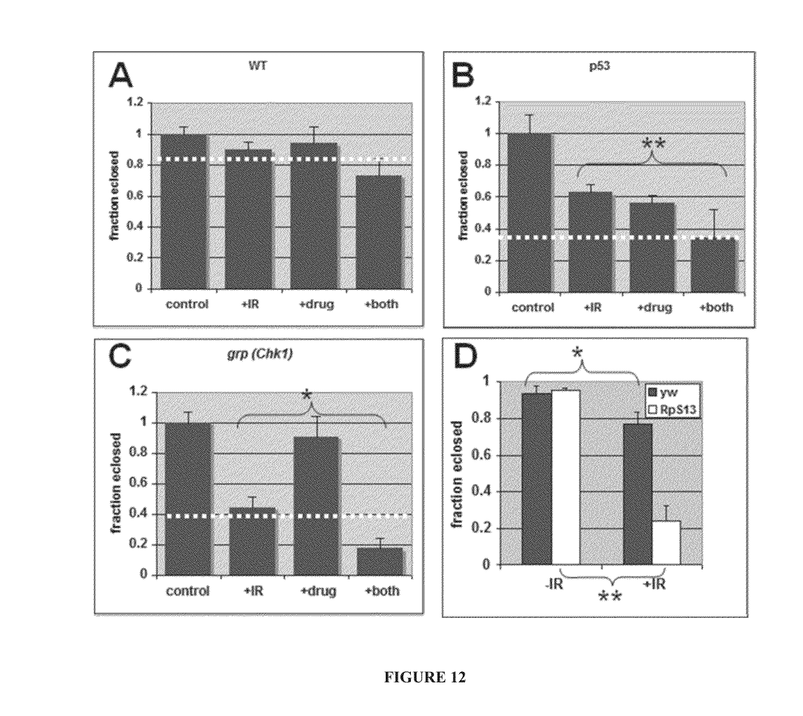

To validate the identification of Bouvardin from the primary screen, the effect of this molecule on Drosophila larvae alone or in combination with radiation were assessed (FIG. 12). The screen was performed using irradiated mutant larvae. Hits from the screen were defined as those that reduced the survival of IRRADIATED larvae compared to the population of drugs being tested (approximately 80 molecules at a time) or compared to DMSO controls in the re-test. A hit could therefore act by enhancing the effect of radiation, or by simply killing larvae as a single agent, or both. To distinguish among these possibilities, hits from the screen were re-tested against larvae with and without irradiation.

In FIG. 12 survival after each treatment is expressed as the fraction of animals that emerged (“eclosed”) as viable adults. The averages have been normalized to the average fraction eclosed in no IR/drug control for the respective genotype. Error bars extend beyond fractional survival of 1.0 simply because of normalization. (A-C) Fraction eclosed for WT and grp and p53 mutants at 16 uM of Bouvardin, with and without IR. The data are averages of 3-8 biological replicates per sample. Dotted line on each graph denotes expected ‘fraction eclosed’ if drug and IR act additively. IR doses were 4000R for WT and grp and 3000R for p53 because of the higher radiation sensitivity of the latter. (D) Fraction eclosed for Minute mutants and yw controls after exposure to 0 (−IR) and 2000R (+IR) of X-rays. N ranged from 86-207 per genotype per treatment, in 3 biological replicates per sample. *=p<0.01; **=<0.001. 100R=1Gy. Error bar=1 standard deviation.

The activity of Bouvardin in Drosophila alone or in combination with ionizing radiation (IR) was assessed. Drug concentrations of 10-20 μM were used to approximate the conditions of the original screen. Third instar larvae were irradiated and cultured on medium containing drug. The survival of larvae was quantified 10 days later by measuring the rate of eclosion into adulthood. Bouvardin increased the killing effect of radiation in a statistically significant manner in grp and p53 mutants but not wild type (compare ‘+IR’ and ‘+both’ samples). The degree of the increase is consistent with the drug and radiation acting in an additive manner on p53 mutants and a synergistic manner on grp mutants. For example, IR alone reduces the eclosion of p53 mutants to 0.63±0.05 and 10 μM of drug reduces it to 0.82±0.15. The combination of Bouvardin and IR reduces eclosion to 0.49, nearly the exact number expected (0.63±0.05×0.82±0.15=0.51±0.10) if the two treatments produced an additive effect. The expected eclosion for additive effect of drug and radiation are denoted with a white dotted line in each graph. For grp mutants, the observed eclosion is lower than the expected for additive effect, suggesting synergy between drug and radiation. Therefore, Bouvardin increases the effect of radiation in either additive or synergistic manner in Drosophila mutants.

### Example 6

Bouvardin shows additive or synergistic effect with radiation on Drosophila larvae. Bouvardin synergizes with radiation to kill Drosophila mutant larvae.

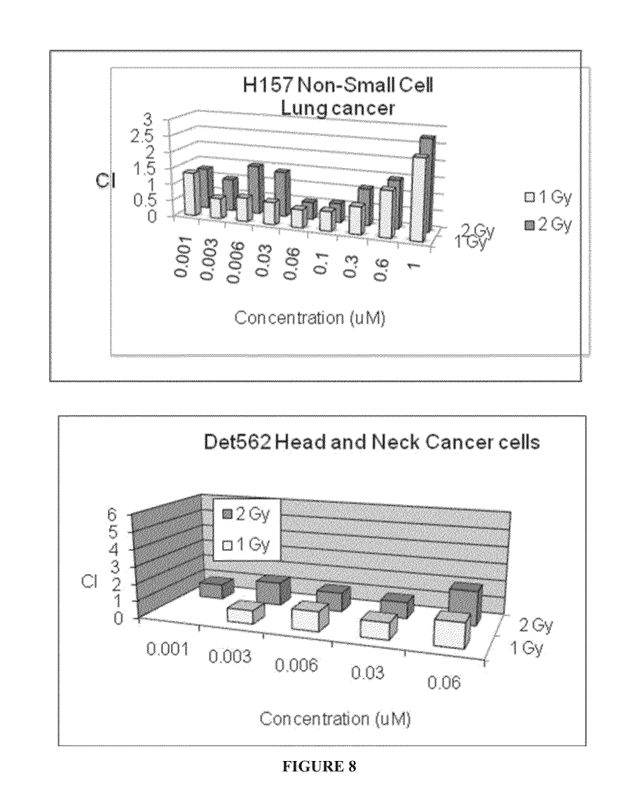

Bouvardin was identified from the NCI library as significantly increasing the effects of radiation on mutant larvae. The effect of bouvardin on mutant and wildtype larvae both alone and in combination with radiation was examined. In wild type flies, there is very little effect with either bouvardin (up to 100 μM) or IR (40Gy) alone. The combination of drug and IR also did not significantly reduce survival to adulthood. In p53 mutants, ionizing radiation (IR) alone reduces the eclosion to 0.62 and 10 mM of drug reduces it to 0.82. The combination of bouvardin and IR reduces eclosion to 0.49, nearly the exact number expected (0.50) if the two produced an additive effect. In grp (Chk1) mutants, bouvardin and IR appear to synergize. FIG. 8 depicts that an observed effect of the combination exceeds additive effect of drug and radiation.

### Example 7

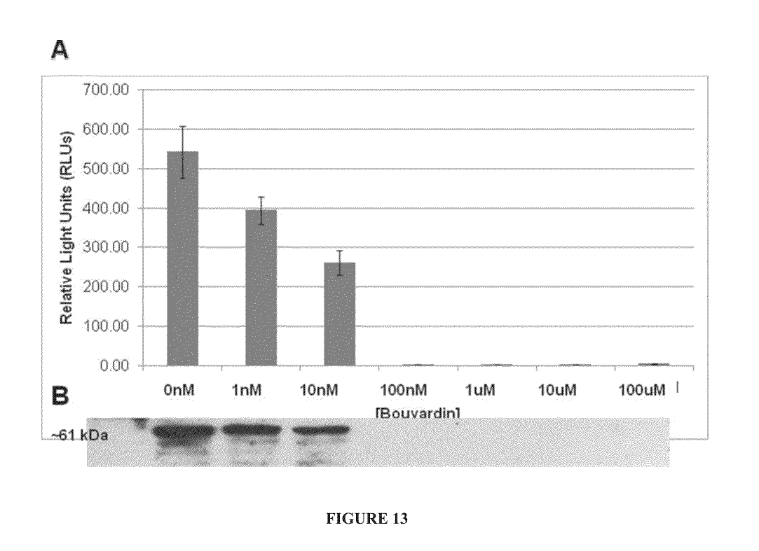

We confirmed that Bouvardin inhibits translation in vitro translation (FIG. 13).

In vitro translation assays were performed in rabbit reticulocytes (Promega) according to manufacturer's instruction, in the presence or absence of Bouvardin at final concentrations shown. Luciferase mRNA provided in the kit was used at a final concentration of 1 ug/ul. The reactions samples were incubated for 15 minutes at 37° C. and quenched by dilution with water. Luciferase activity was measured using a plate reader Multi-Mode Microplate Reader (Synergy 2 by BioTek) immediately after luciferase substrate addition. Samples were also subjected to electrophoresis on SDS-polyacrylamide gels and immunoblotted with goat polyclonal α-luciferase primary antibodies (Promega) and donkey anti-goat IgG, HRP secondary antibodies (Promega). The blots were visualized using chemiluminescence detection (Pierce). (A) Luciferase activity at different concentrations of Bouvardin. Error bars show SEM from triplicate samples. (B) Luciferase protein levels in corresponding samples.

Nonetheless, drugs can have off-target effects. To more directly test the idea that inhibition of translation increases sensitivity to radiation, the radiation sensitivity of two Drosophila Minute mutants and wild type were compared. Minute genes are essential genes that encode structural components of the ribosome. Minute heterozygotes with decreased ribosomal protein L36 or S13 gene dosage are more sensitive to radiation than yw controls (FIG. 12D for RPs13). These results support the idea that optimal translation capacity is needed for radiation survival.

### Example 8

Bouvardin synergizes with radiation on human cancer cells.

The observation that Bouvardin enhanced the effect of radiation on Drosophila larvae led us to question whether this effect would translate to a mammalian system. In order to determine what cell lines should be used to create tumor xenografts in mice, MTT assays for cell viability were used first to test the effects of Bouvardin alone and in combination with radiation in human cancer cells. MTT assays measure cell viability by measuring mitochondrial enzyme function. We used 17 cell lines including Head and Neck Squamous Cell Carcinoma and non-small cell lung cancer lines.

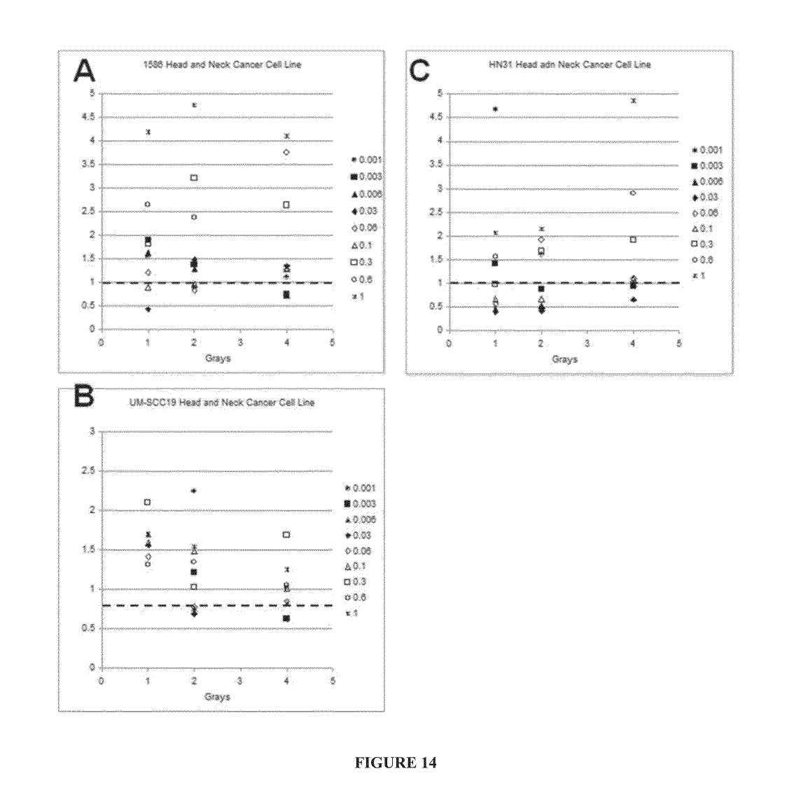

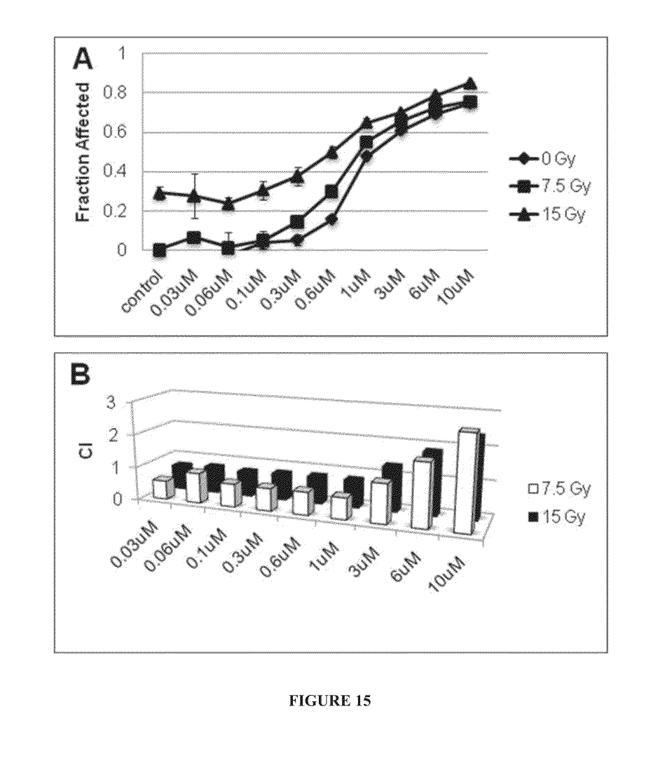

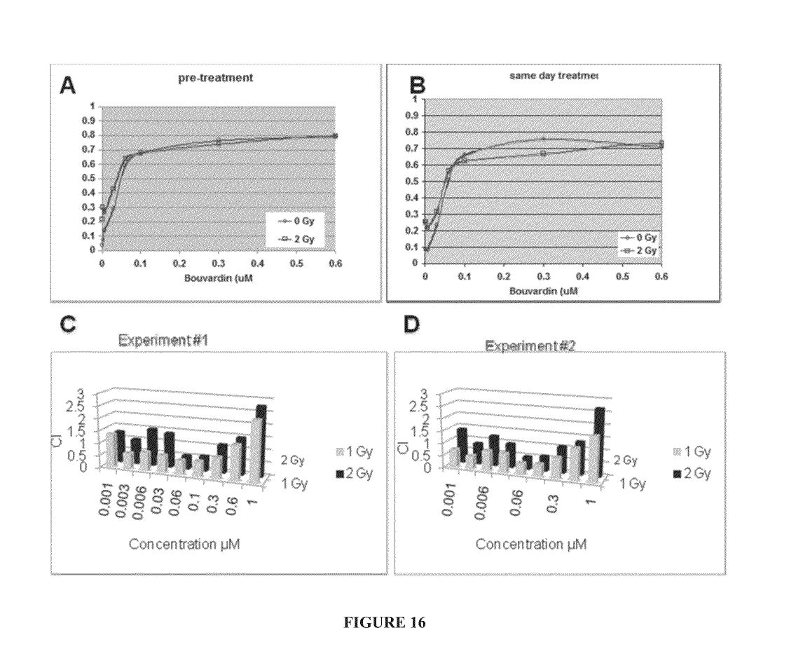

Bouvardin and radiation synergize in most, but not all of these cell lines. A representative sample of these data, from 7 cell lines, are in FIGS. 14 and 15. The data for non-small cell lung cancer cell line (H157) is depicted in FIG. 16. This line was chosen for further study because of an observed synergy over a wide range of doses between Bouvardin and radiation in this cell line and because it is known to be amenable to xenografting in mice.

In FIG. 16 MTT assays were used to determine the effect of Bouvardin in combination with IR. Cells were either treated with drug for 24 hr prior to irradiation (A and C, ‘pre-treatment’) or drug and radiation were applied on the same day (B and D, ‘same day treatment’). (A & B) Fraction of cells killed or otherwise eliminated (Fraction Affected) at different doses of Bouvardin and 0 or 2 Gy of IR. (C & D) Combination Index (CI) data from two experiments with pre-treatment protocol, to illustrate reproducibility. CIs below 1 indicate synergy. Bouvardin synergizes with ionizing radiation under several experimental conditions.

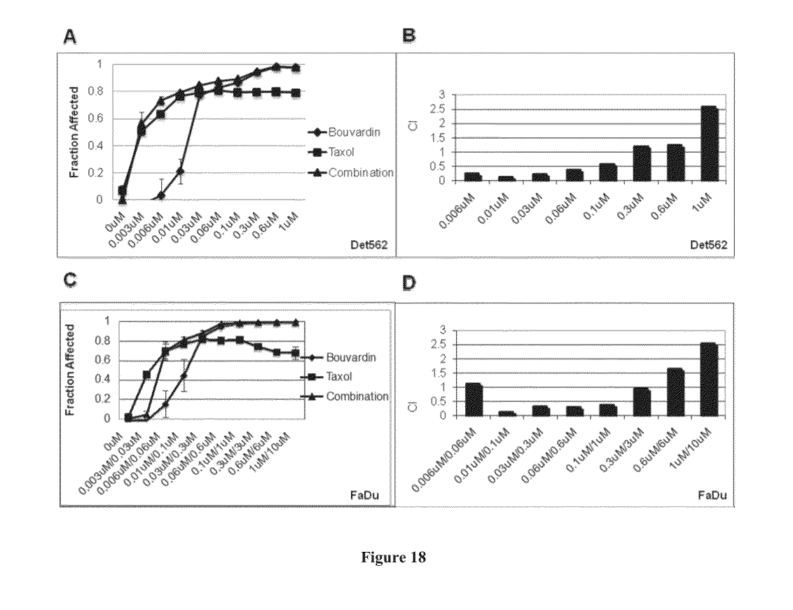

In the absence of radiation, the drug has an IC50 (Fraction Affected, Fa=0.5) in the nM range (FIGS. 18A and B, ‘0 Gy’). Fraction Affected is determined by the formula, 1−(x/y), where x is the MTT signal for the experimental sample and y is the MTT signal for the un-treated control. For example, if the MTT signal in the experimental is 30% of the MTT signal in the control, fraction affected is 0.7. Radiation increased the Fa in a dose-dependent manner in the absence of drug. The addition of the drug further increased the Fa for each dose of radiation. This happened whether the drug was added 24 hr before irradiation (pre-treatment, FIGS. 16A,C) or concurrently (same day treatment, FIGS. 16B,D).

The efficiency of Bouvardin in increasing the effect of radiation on cancer cells is measured in terms of Combination Index (CI) as determined by CalcuSyn software according to the method of Chou and Talalay. This method is widely used in biomedical literature and it has been applied to combinations of antitumor drugs. CI values are calculated from Fa values for multiple drug and radiation doses such as those shown in FIGS. 16 A and B. CI computation takes into account the shape of the dose-response curve, the maximal effect, the intercept, and the IC50 values for drug at each dose of radiation and IC50 for radiation at each dose of drug. CI values can be above, below or equal to 1, indicating antagonizing, synergistic or additive effects respectively. These results show that Bouvardin synergizes with 1-2 Gy of radiation at many concentrations between 0.006 μM and 0.3 μM (FIG. 16).

For FIG. 18 CellTiter-Glo® Luminescent Cell Viability Assays (Promega G7570) were used to determine the effect of Bouvardin in combination with Taxol on Det562 (A and B) and FaDu (C and D) cells. In (A) and (B), the concentrations shown are those of each drug. For example, 0.1 uM in=0.1 uM of Bouvardin and 0.1 uM of taxol. In (C) and (D), the concentrations are those of Bouvardin and taxol respectively. For example, 0.3 uM/3 uM means 0.3 uM of Bouvardin and 3 uM of taxol. (A and C) Fraction of cells killed or otherwise eliminated (Fraction Affected) at different doses of Bouvardin and taxol. (B and D) Combination Index (CI) data for Det562 and FaDu cells are shown. CI below 1 indicates synergy. On Det652 cells, Bouvardin shows synergy in combination with taxol at doses ranging from 0.006 uM-0.1 uM. On FaDu cells, Bouvardin shows synergy in combination with taxol at doses ranging from 0.01 uM-0.3 uM for Bouvardin and 0.1 uM-3 uM for Taxol. Error bar=±1STD.

### Example 9

Bouvardin synergizes with radiation on H157 and UMSCC2B cells.

MTT assays were used to test the effects of bouvardin alone and in combination with radiation on numerous cell lines. Bouvardin and radiation synergize in non-small cell lung cancer cell line (H157) and head and neck squamous cell carcinola line (UMSCC2B) over a wide range of concentrations between bouvardin and radiation. FIG. 8 depicts this synergy in these two cell lines. The efficiency of bouvardin in sensitizing cancer cells to radiation is measured in terms of Combination Index (CI) as determined by Calcusyn software. CI values are either above, below or equal to 1, indicating antagonizing, synergistic or additive effects respectively. “Gy” in FIG. 8 stands for “Gray,” a radiation unit.

### Example 10

Bouvardin enhances the effect of IR on H157 cells in clonogenic assays and xenografts.

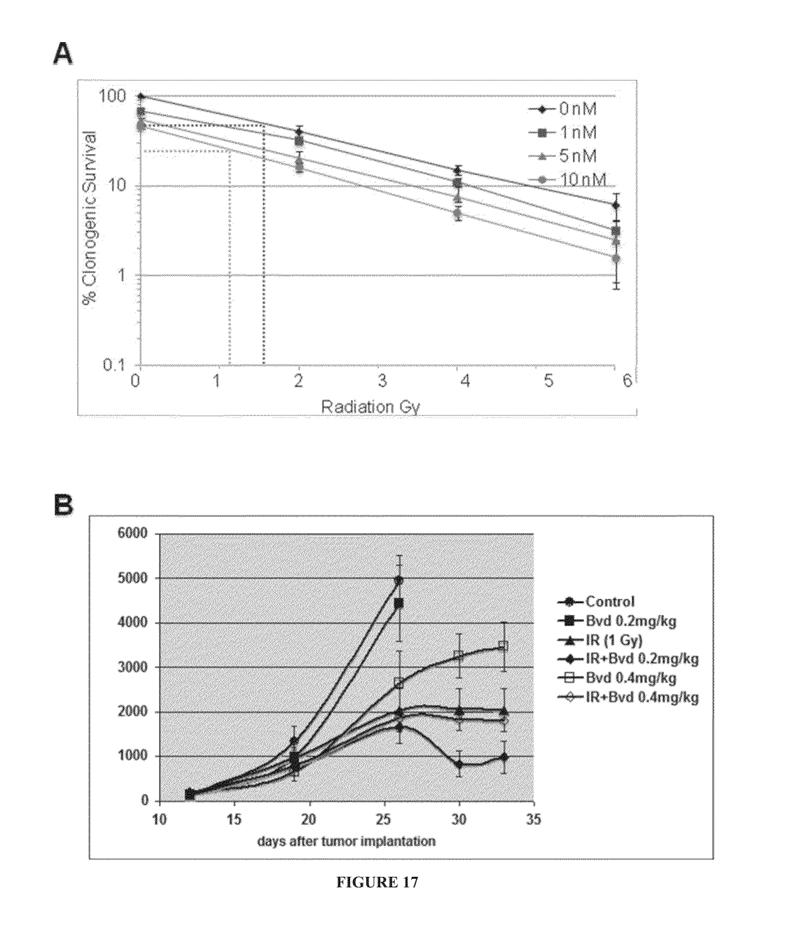

To assess the effect of Bouvardin, another measure of cell survival, namely, clonogenic assays, was employed. Bouvardin also increased the effect of radiation in reducing the clonogenicity of H157 cells (FIG. 17A). For example, IC50 for IR at 0 and and 10 nM of Bouvardin, extrapolated from the graph in FIG. 17A, are approximately 1.5 Gy and 1.0 Gy (dark and light dotted lines respectively).

The effectiveness of Bouvardin, IR and the combination of the two was next assessed in H157 tumor xenografts (FIG. 17B). We find that drug alone administered at 0.2 mg/kg had little effect and 1 Gy of radiation alone showed partial tumor control. Importantly, the combination of drug and radiation showed improved control of tumor volume in a statistically significant manner (p=0.0387 on day 30; upaired two tailed t-test). Similar results were obtained in a repeat experiment (not shown). These data demonstrated a potential use for Bouvardin in cancer therapy, namely in combination with radiation. Bouvardin enhances the effect of radiation in Drosophila mutant larvae, human cancer cells and tumor xenografts in mice. The choice of drug concentration would be critical. At 0.4 mg/kg, the drug on its own shows tumor control but did not enhance the effect of radiation. Similar effects of higher drug dose were seen in two different experiments (only one shown here).

In FIG. 17 (A) Clonogenic assays were used to confirm the effects of Bouvardin in combination with radiation in H157 cells. Cells were plated in 6 well plates at 1,000 cells per well. 24 hours later, various doses of (1 nM, 5 nM and 10 nM) of Bouvardin were added with media alone as a control. 24 hours after the addition of Bouvardin, the cells were irradiated at 0, 2, 4 and 6 Gy. Colonies were allowed to form for 2 weeks and then counted. The results are shown as a percentage of control (no drug, no IR). Error bar=±1SEM. (B) The effect of Bouvardin (Bvd) and radiation on H157 xenografts in athymic mice. Bouvardin treatment began when tumors had reached approximately 200 mm3. Bouvardin was administered intraperitonealy twice a week, and radiation was administered 24 hr after each drug administration Animals in control and drug-only groups had tumors that were too large and had to be sacrificed on day 26. Treatment continued for the other two groups for one more week. The difference in tumor volume between combination-treated group and radiation only group was statistically significant at day 30 (p=0.0387 on day 30; upaired two tailed t-test). The difference in tumor volume between combination-treated group and radiation only group remains on day 33 but is less significant (p=0.0967; upaired two tailed t-test). N=10 per group. The data points indicate mean tumor volumes ±1SEM.

### Example 11

Bouvardin synergizes with Taxol in human cancer cells.

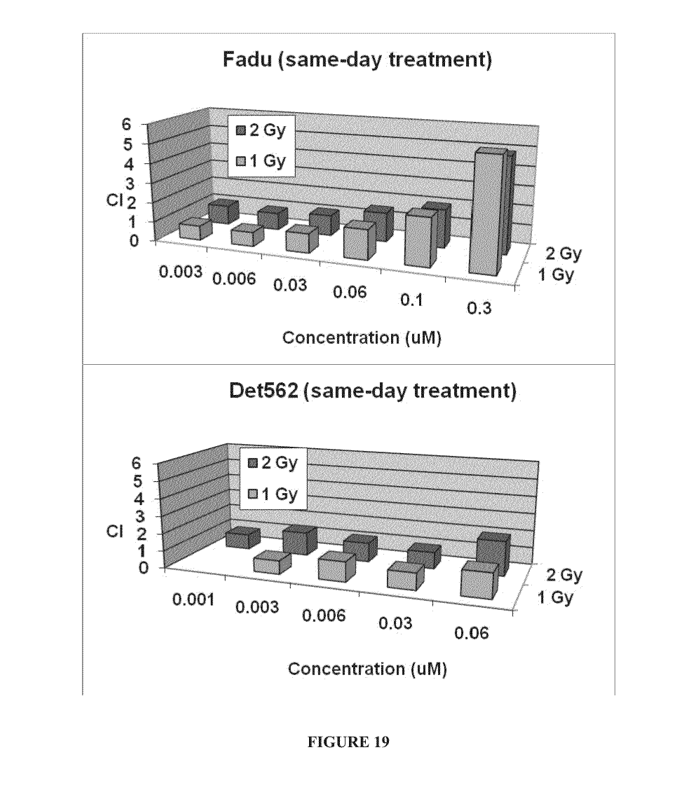

The therapeutic effect of radiation is thought to result from cell killing that follows radiation damage. Synergy between Bouvardin and radiation, we propose, is based, at least in part, on the ability of Bouvardin to prevent protein synthesis needed to recover from radiation-induced damage. If this was true, we may see synergy between Bouvardin and other agents that promote cell killing. To test this idea, we investigated whether Bouvardin also synergizes with a chemotherapeutic drug on two human head and neck cancer cell lines, Det562 and FaDu (FIG. 28). Det562 cells are more sensitive to Taxol alone than FaDu cells; this necessitates the use of lower Taxol concentrations on the former. Nonetheless, Bouvardin shows synergy with Taxol over a wide range of concentrations in both cell lines. In the same cell lines, Bouvardin shows a wide range of effects, synergistic to antagonistic, in combination with IR (FIG. 19). Synergy was seen for up to 0.3 μM Bouvardin when combined with taxol (FIG. 18) but only at concentrations lower than 0.06 μM when combined with radiation (FIG. 19).

The interaction is synergistic in several cell lines and that Bouvardin can enhance the effect of IR in clonogenic assays and in xenograft models. The concentration of the drug can affect the nature of its interaction with IR both in cells and in xenografts. 0.2 mg/kg of Bouvardin had little effect on its own in the xenograft model but enhanced the effect of IR. 0.4 mg/kg of drug, on the other hand, had effect on its own but did not enhance the effect of IR. If distributed homogeneously, 0.2 and 0.4 mg/kg would correspond to approximately 0.3 and 0.6 μM. In cell-based assays, 0.3 and 0.6 μM of drug acted, respectively, synergistically and additively/antagonistically with radiation, similar to the outcome in xenograft experiments (FIG. 17).

Bouvardin is found to synergize with another standard therapy, taxol, in cell-based assays. These results suggest that translation inhibitors as a class of molecules may show clinical efficacy in combination with other anti-cancer therapies.

Three lines of data suggest strongly, however, that anti-translation effect of Bouvardin is key. These are: IC50 for Bouvardin in cells and in in vitro translation reactions (FIG. 13) are in the similar, nM range; Bouvardin and Minute mutations both increase the effect of radiation on Drosophila larvae; and, another translation inhibitor, Streptovitacin A, behaves similarly to Bouvardin in the same cell line (FIG. 15).

For FIG. 15 CellTiter-Glo® Luminescent Cell Viability Assay (Promega G7570) were used to determine the effect of Streptovitacin (concentrations in uM) in combination with ionizing radiation (in Gy, X-axis). The drug was added 24 hr before irradiation. (A) Faction affected (Fa) determined by the following formula 1−(x/y) where x is the experimental sample and y is the no Bouvardin/no drug control) (B) Corresponding CI values. Cells were irradiated in a Faxitron X-ray cabinet x-ray system (Lincolnshire, Ill.) set at 115 kV and 5 mA (producing 5.33 Rads/sec or 3.2 Gy/min) Note that this X-ray machine, produces a higher dose rate (lower exposure time) than the X-ray machine used on H157 cells in FIG. 18. Even with the same settings, higher IR doses are needed to achieve the same Fa. More important, combinatorial effects with drugs are similar if not identical on the two machines. The drug and radiation synergize at most combinations tested.

For FIG. 19 MTT assays were used to determine the effect of Bouvardin (concentrations in uM) in combination with ionizing radiation (in Gy). The data from same-day treatment of IR and Bouvardin are shown, for comparison with taxol-Bouvardin combination that were also applied on the same day. In general, CI values were lower for FaDu cells than for Det562 cells in two separate experiments (only one set is shown). On Det562 cells, CI values were about 1 for most combinations. CI value for Det562 cells at 0.001 uM/1Gy and at >0.06 uM are not included in the graph because the values are >10 and would have made smaller values harder to see. On FaDu cells, CI values were below 1 at concentrations of 0.03 or lower, were about 1 for 0.06 uM drug, and were over 1 at higher concentrations.

### Example 12

A bouvardin derivative is as effective as bouvardin in Drosophila.

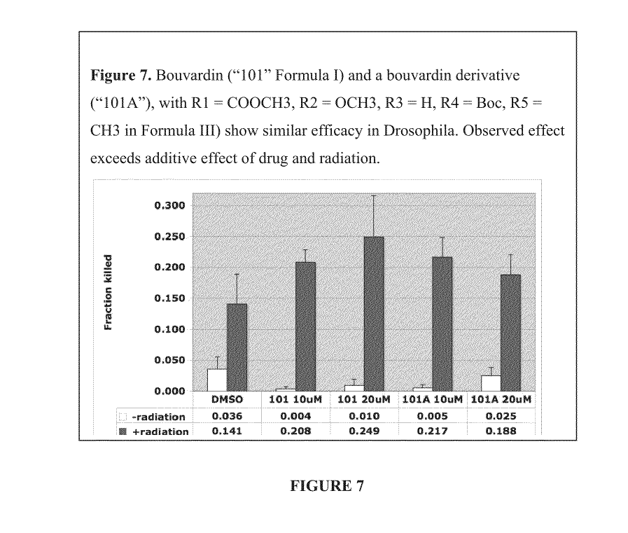

A seven-step synthesis was designed to obtain bourvardin derivatives, as shown in FIG. 5. Bouvardin derivatives are depicted in FIG. 4 as a generic Formula III, with the specific derivative used in this example having R1=COOCH3, R2=OCH3, R3=H, R4=Boc, R5=CH3. This derivative is as effective as bouvardin in sensitizing flies to ionizing radiation, commonly used for radiation therapy for human cancers. FIG. 7 shows that an observed effect exceeds additive effect of drug and radiation.

### Example 13

### Drosophila Assays

Wild type flies were of the Sevelin stock. Mutants have been described before. p535A-1-4 results from targeted deletion of the gene, and is maintained and used as homogygotes. grpl (Chk1 mutants) results from a p-element insertion and is a genetic null. Homozygotes of the latter were identified by the lack of GFP marker on the balancer chromosome. Minute mutants were of the genotypes y[1]w[*]; P[w[+mC]=lacW]RpS131/CyO and P[lacW]RpL36G0471 w67c23/FM7a. Because Minute mutants were in yw or w background, y1w1 (Bloomington stock #1495) was used as ‘wild type’ control in these experiments.

Feeding stage 3rd instar larvae were irradiated as previously described (2). Briefly, 120 hr old larvae were rinsed to remove food and passed through sizing sieves to obtain animals of uniform size. Larvae were placed in a Petri dish and irradiated using a TORREX X-ray generator, set at 115 kV and 5 mA (producing 2.4 Rads/sec or 1.44 Gy/min) Irradiated larvae were then cultured on cornmeal-agar media (2) containing drug or DMSO carrier. In all experiments using human cells, cells in 96-well plates were irradiated with a RS2000 Biological Irradiator (Rad Source Technologies, Inc.) delivering 1 Gy/min.

### Example 14

### Cell Lines

Head and Neck (HNC) and non-small cell lung carcinoma (NSCLC) cell lines were used. H157 cells are maintained in RPMI (“Roswell Park Memorial Institute”) medium with 10% heat-inactivated fetal bovine serum (FBS; Hyclone, Logan, Utah), and Detroit562 and FaDu are maintained in DMEM (Dulbecco's Modified Eagle's Medium) with heat-inactivated 10% FBS. These cell lines were maintained in a humidified incubator with 5% CO2. Cell lines were authenticated by DNA fingerprinting during the course of the experiments.

### Example 15

### Cell Growth Assays

The growth inhibitory effects of Bouvardin with ionizing radiation (IR) were evaluated using a modified tetrazolium salt (MTT) assay. In the MTT assay, 1,000 to 2,000 viable cells were plated in 100 uL of growth medium in 96-well plates (Corning, Ithaca, N.Y.). Following an overnight incubation, drug was added in varying concentrations and the plates were irradiated on the same day (co-treatment) or 24 hr later (pre-treatment) and incubated for 6 to 7 days. The tetrazolium salt was added at a concentration of 0.4 mg/mL to each well following the 6- to 7-day treatment. The plates were incubated with the salt for 4 hours at 37° C. At 4 hours, the medium was aspirated off, leaving the dark blue formazan product at the bottom of the wells. The reduced MTT product was solubilized by adding 100 mL of 0.2 N HCl in 75% isopropanol, 23% MilliQ water to each well. Thorough mixing was done using a Titertek multichannel pipetman. The absorbency of each well was measured using an automated plate reader (Molecular Devices, Sunnyvale, Calif.). All experiments were done in triplicate.

### Example 15

The growth inhibitory effects of Bouvardin with Taxol were evaluated using CellTiter-Glo® Luminescent Cell Viability Assay (Promega G7570). The CellTiter-Glo® Reagent lyses cells and generates a luminescent signal proportional to the amount of ATP present. In this assay, 4,000 viable cells were plated in 100 uL of growth medium in 96-well plates (Corning). Following an overnight incubation, both drugs were added in varying concentrations and incubated for 6 days. 100 uL of CellTiter-Glo® Reagent was added to each well. Plates were incubated with mixing at room temperature for 30 minutes. Luminescence of each well was measured using an automated plate reader.

### Example 16

### Clonogenic Assays

The survival of cells treated with Bouvardin and IR was measured by performing standard clonogenic assays. 1,000 cells were added per well in 2 ml of media in 6 well plates. Following an overnight incubation, varying concentrations of drug were added to the wells. 24 hrs later, the plates were irradiated at varying doses and incubated for ˜2 weeks or until colonies were large enough to count. The colonies were then stained with crystal violet (1% in methanol), rinsed 3× with water, and colonies with greater than 50 cells were counted using a dissection microscope. Fraction survival was calculated as a percentage of the control (no drug, no IR).

### Example 17

### Xenograft Assays

Six to 8-week-old female nude athymic mice were purchased from Harlan Laboratories and housed in a pathogen-free facility approved by the American Association for the Accrediation of Laboratory Animal Care and met all current regulations and standards of the U.S. Department of Agriculture, the U.S. Department of Health and Human Services and the National Institutes of Health Animal procedures were carried out in accordance with a protocol approved by the Institutional Animal Care and Use Committee of the University of Colorado. Cancer cells were grown to 80% confluence and harvested by trypsinization. Trypsin was neutralized with complete medium containing FBS. Cells were washed 3× with RPMI (no FBS) and resuspended in unsupplemented RPMI to a concentration of 2×106 per 100 μl. Cells were mixed 1:2 with matrigel (BD Biosciences #354234) and 1×106 cells/mouse (100 μl) were injected subcutaneously into the rear flanks of athymic nude mice. Mice bearing tumors with a volume of ˜200 mm3 were randomly assigned to treatment groups of 8-10 animals each (control, drug alone, radiation alone or the combination). Each week, animals received drug diluted in saline (or saline carrier) by intraperitoneal injection on Mondays and Wednesdays, and a 2 Gy radiation dose on Tuesdays and Thursdays was delivered by X-ray irradiation. Mice were anesthetized with ketamine/xylazine before radiation and positioned under a lead shield such that only the tumor-bearing leg was exposed. Mice were treated for 3 weeks, and tumor growth was measured until volume exceeded 2 cm3, when animals were euthanized. Tumor volume was measured on Tuesdays and Fridays. Tumor volume was calculated using the formula V=a2×b/2, where a and b are the smallest and largest tumor diameters, respectively, as determined using calipers.

### Example 18

MTT assays were used to determine the effect of Bouvardin (concentrations in uM) in combination with ionizing radiation (in Gy, X-axis). FIG. 14 depicts CI values for Bouvardin and radiation on 3 cell lines. MTT assays were used to determine the effect of Bouvardin (concentrations in uM) in combination with ionizing radiation (in Gy, X-axis). Combination Index data from 3 different Head and Neck Cancer cell lines are shown. Black dashed lines represent a CI value of 1. CI values below 1 indicate synergy. 1586 and UM-SCC19 were pre-treated whereas HN31 was treated on the same day.

Combination Index data from 3 different Head and Neck Cancer cell lines are shown. Black dashed lines represent a CI value of 1. CI values below 1 indicate synergy. 1586 and UM-SCC19 were pre-treated whereas HN31 was treated on the same day.

TABLE 2
Fraction Affected and CI values for Bouvardin on 3 additional
cell lines. A table representing the fraction affected and CI
values for each dose of Bouvardin and IR for the three cell lines
shown in FIG. 14. Fraction affected is determined by the
following formula 1 − (x/y) where x is the experimental
sample and y is the no Bouvardin/no drug control.

| | Fraction | | | Combination | | |
|---|---|---|---|---|---|---|
| Bouvardin | Affected | | | Index | | |
| (uM) | 1 Gy | 2 Gy | 4 Gy | 1 Gy | 2 Gy | 4 Gy |
| | A. 1586 | | | | | |
| 0.001 | 0.254 | 0.23 | 0.503 | 1.553 | 0.903 | 1.112 |
| 0.003 | 0.264 | 0.303 | 0.51 | 1.898 | 1.383 | 0.753 |
| 0.006 | 0.301 | 0.371 | 0.634 | 1.626 | 1.285 | 0.732 |
| 0.01 | 0.45 | 0.518 | 0.604 | 0.433 | 1.482 | 1.348 |
| 0.03 | 0.537 | 0.628 | 0.624 | 1.211 | 0.841 | 1.284 |
| 0.06 | 0.584 | 0.677 | 0.813 | 0.898 | 0.962 | 1.284 |
| 0.1 | 0.607 | 0.68 | 0.712 | 1.815 | 3.214 | 2.639 |
| 0.3 | 0.682 | 0.731 | 0.708 | 2.656 | 2.382 | 3.756 |
| 0.6 | 0.688 | 0.743 | 0.745 | 4.194 | 4.765 | 4.11 |
| 1 | 0.254 | 0.23 | 0.503 | 1.553 | 0.903 | 1.112 |
| | B. UM-SCC19 | | | | | |
| 0.001 | 0.143 | 0.28 | 0.386 | 13.885 | 2.245 | 1.023 |
| 0.003 | 0.227 | 0.366 | 0.466 | 5.143 | 1.22 | 0.627 |
| 0.006 | 0.354 | 0.448 | 0.461 | 1.696 | 0.762 | 0.82 |
| 0.01 | 0.395 | 0.49 | 0.521 | 1.561 | 0.694 | 0.619 |
| 0.03 | 0.44 | 0.511 | 0.515 | 1.414 | 0.777 | 0.848 |
| 0.06 | 0.431 | 0.444 | 0.498 | 1.587 | 1.482 | 1.01 |
| 0.1 | 0.437 | 0.518 | 0.473 | 2.102 | 1.027 | 1.691 |
| 0.3 | 0.523 | 0.524 | 0.558 | 1.313 | 1.347 | 1.052 |
| 0.6 | 0.532 | 0.545 | 0.573 | 1.704 | 1.546 | 1.251 |
| 1 | 0.143 | 0.28 | 0.386 | 13.885 | 2.245 | 1.023 |
| | C. HN31 | | | | | |
| 0.001 | 0.165 | 0.292 | 0.478 | 4.673 | 1.605 | 0.989 |
| 0.003 | 0.362 | 0.463 | 0.535 | 1.405 | 0.877 | 0.93 |
| 0.006 | 0.574 | 0.602 | 0.559 | 0.462 | 0.517 | 1.005 |
| 0.01 | 0.731 | 0.747 | 0.73 | 0.401 | 0.419 | 0.657 |
| 0.03 | 0.753 | 0.782 | 0.717 | 0.565 | 0.471 | 1.099 |
| 0.06 | 0.778 | 0.785 | 0.756 | 0.663 | 0.673 | 1.086 |
| 0.1 | 0.825 | 0.789 | 0.784 | 0.97 | 1.673 | 1.914 |
| 0.3 | 0.837 | 0.826 | 0.8 | 1.563 | 1.911 | 2.897 |
| 0.6 | 0.85 | 0.849 | 0.797 | 2.067 | 2.148 | 4.862 |
| 1 | 0.165 | 0.292 | 0.478 | 4.673 | 1.605 | 0.989 |

### Example 19

To a solution of Compound 1 (200 mg, 0.357 mmol, 1.0 eq) in anhydrous DMF (4 ml) was added NaH (17.14 mg, 0.4285 mmol, 1.2 eq, 60% in mineral oil) at 0° C. under the protection of N₂ and the reaction mixture was stirred at 0° C. for 30 mins. Then to the reaction mixture was added CH₃I (507 mg, 3.57 mmol, 10 eq) under the protection of N₂ and the reaction mixture was stirred at 0° C. for 3 hrs. TLC (PE:EA=2:1) showed the reaction was completed. Saturated aq. NH₄Cl (4 ml) was added at 0° C. and the reaction mixture was extracted with EA (4 ml×4). The organic layers were combined and dried over anhydrous Na₂SO₄, filtered and concentrated. The residue was purified by column chromatography on silica gel with Petro ether: Ethyl acetate=2:1 to give crude Compound 2 (150 mg, 73% yield) which was racemized. It needs further separation by chiral SFC to get diastereomers of compound 2.

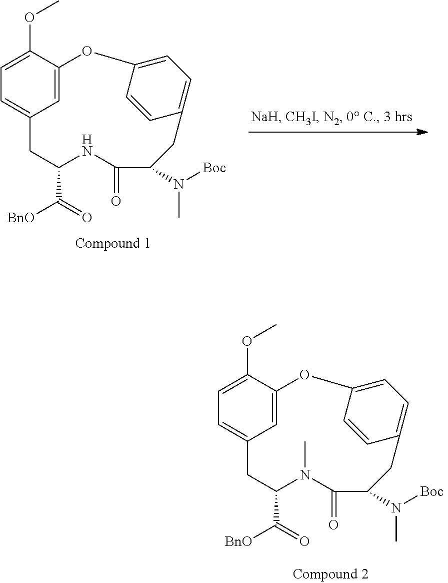 [SMILES: COc1ccc2cc1Oc1ccc(cc1)C[C@H](N(C)C(=O)OC(C)(C)C)C(=O)N[C@H](C(=O)OCc1ccccc1)C2 ; COc1ccc2cc1Oc1ccc(cc1)C[C@H](N(C)C(=O)OC(C)(C)C)C(=O)N(C)[C@H](C(=O)OCc1ccccc1)C2]

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for treating a disorder in a subject comprising
administering to a subject a sub-therapeutic amount of bouvardin.

2. The method of claim 1, further comprising method for treating a disorder in a subject comprising:
administering to the subject an effective amount of radiation therapy.

3. The method of claim 1, further comprising administering to the subject an effective amount of chemotherapeutic composition.

4.-13. (canceled)

14. The method of claim 1, wherein the disorder is cancer, an immune disorder, or diabetes.

15. The method of claim 14 wherein the cancer is a cancer in tissues of head and neck, a cancer in tissues of the lung, or a cancer of the lymphatic system.

16.-19. (canceled)

20. The method of claim 1, wherein the disorder is a neurological disorder associated with abnormal protein accumulation.

21. The method of claim 2 wherein the radiation therapy comprises exposing the subject to ionizing radiation, exposing the subject to particles from a radioactive substance, or exposing the subject to radiation from an external source.

22. (canceled)

23. (canceled)

24. The method of claim 2, wherein the radiation therapy is for curative, adjuvant, neoadjuvant, therapeutic or palliative purposes.

25. The method of claim 2 wherein the radiation therapy is given at a dosage of 20 Gy to 80 Gy total, fractionated into smaller doses over a course of treatment that may last several weeks.

26. (canceled)

27. (canceled)

28. The method of claim 3 wherein the chemotherapy composition comprises a taxane.

29. (canceled)

30. (canceled)

31. The method of claim 3 wherein chemotherapy composition comprises a platinum-based chemotherapy drug.

32. The method of claim 3 wherein chemotherapy composition comprises doxorubicin or a derivative of doxorubicin.

33. The method of claim 1, wherein the bouvardin is administered orally, intravenously, or by local injection.

34. The method of claim 1, wherein the bouvardin is administered at a concentration of 0.01 to 10 mg/kg.

35.-49. (canceled)

50. A composition for use in the method of claim 3, wherein the composition further comprises bouvardin and a chemotherapeutic agent.

51.-55. (canceled)

56. A method comprising administering to a patient a composition, wherein a) the patient is in need of treatment, the patient has had radiotherapy, the patient has been diagnosed with cancer, and the patient is a human; and b) the composition comprises bouvardin.

57. (canceled)

58. The method of claim 56 wherein the amount of bouvardin in the composition would be sub-therapeutic if administered alone.

59. The method of claim 56 wherein the amount of bouvardin administered is less than 0.01 mg/kg, less than 0.02 mg/kg, less than 0.05 mg/kg, less than 0.1 mg/kg, less than 0.5, less than 1.0 mg/kg, less than 1.5 mg/kg, or less than 2.0 mg/kg.

60. A method comprising:
a) obtaining a sample from a tumor from a patient with cancer,
b) assessing the level of protein translation said tumor,
wherein the level of protein translation can be used to determine whether the patient should be administered bouvardin.

61. The method of claim 60 wherein the patient has been treated with radiotherapy.
